(19) United States
(12) Patent Application Publication
(10) Pub. No.: US 2024/0325537 A1
(43) Pub. Date: Oct. 3, 2024

(54) CELLULAR THERAPIES FOR CANCER BY INHIBITION OF MONOCARBOXYLATE TRANSPORTER 11

(71) Applicant: University of Pittsburgh - Of the Commonwealth System of Higher Education, Pittsburgh, PA (US)

(72) Inventors: Greg M. Delgoffe, Pittsburgh, PA (US); Ronal Peralta, Pittsburgh, PA (US)

(73) Assignee: University of Pittsburgh - Of the Commonwealth System of Higher Education, Pittsburgh, PA (US)

(21) Appl. No.: 18/580,954

(22) PCT Filed: Jul. 19, 2022
(86) PCT No.: PCT/US2022/037633

§ 371 Date: Jan. 19, 2024

Related U.S. Application Data
(60) Provisional application No. 63/223,453, filed on Jul. 19, 2021.

(51) Int. Cl. A61K 39/00; A61K 45/06; A61P 35/00; C07K 14/725; C12N 5/0783; C12N 15/113

(52) U.S. Cl. CPC A61K 39/464412; A61K 39/4611; A61K 39/4644; A61K 39/464404; A61K 39/464406; A61K 39/464417; A61K 39/464424; A61K 39/464466; A61K 39/464468; A61K 39/46447; A61K 39/464474; A61K 39/464482; A61K 39/464494; A61K 45/06; A61P 35/00; C07K 14/7051; C12N 5/0636; C12N 15/113; C12N 2310/14; C12N 2310/20; C12N 2310/531; C12N 2501/2302; C12N 2510/00

(57) ABSTRACT

The present disclosure provides compositions, including modified peripheral blood mononuclear cells (PBMCs) with reduced expression of Slc16a11 and/or reduced activity of MCT11. Also provided are siRNAs and gRNAs targeting Slc16a11. Methods of using the disclosed compositions in the treatment of cancer are also provided.

### CROSS REFERENCE TO RELATED APPLICATIONS

This claims the benefit of U.S. Provisional Application No. 63/223,453, filed Jul. 19, 2021, which is incorporated by reference herein in its entirety.

### FIELD

This disclosure relates to immunotherapies, particularly compositions and methods of preventing or reducing T cell exhaustion and uses thereof for treating cancer or improving immunotherapy.

### BACKGROUND

The programmed cell death 1 (PD-1) receptor is a checkpoint receptor mainly expressed on mature cytotoxic T lymphocytes. Cancer cells often express PD-1 ligands, such as PD-L1 and PD-L2, leading to immune tolerance of cancerous cells. Certain cancer therapies target PD-1 or its ligands to reduce immune tolerance, thereby increasing T cell mediated elimination of cancerous cells. However, only a subset of patients respond to this so-called PD-1 blockade. A potential factor limiting efficacy is the development of T cell exhaustion, an alternative differentiation fate of T cells to a dysfunctional state. Exhaustion limits the capacity of T cells to target tumor cells or respond to immunotherapy. Thus, increasing effector function of T cells, or increasing resistance to exhaustion, may be useful for improving patient response to various cancer immunotherapies, such as PD-1 blockade.

### SUMMARY

MCT11 is a transporter protein. It is shown here that MCT11 is present on the surface of terminally exhausted T cells, especially those that infiltrate tumors. Decreasing expression of MCT11 in shown to slow development of T cell exhaustion and helps retain anti-tumor functionality. Thus, MCT11 activity may contribute to T cell exhaustion. Without being bound to any particular theory, MCT11 may transport monocarboxylates, such as lactic acid. Thus, MCT11 mediated uptake of lactic acid (or another MCT11 substrate) may reduce anti-tumor function of T cells. Based on these observations, methods of generating PBMCs, such as T cells, with reduced expression of Slc16a11, reduced activity of MCT11, or both, to reduce or prevent T cell exhaustion in PBMCs or T cells used for cancer treatment, such as immunotherapy, are provided.

Provided herein are modified peripheral blood mononuclear cells (PBMCs) with reduced expression of Slc16a11, reduced activity of MCT11, or both. In some embodiments, the modified PBMC includes an agent that reduces Slc16a11 expression, for example, an inhibitory RNA (RNAi) or guide RNA (gRNA) specific for a Slc16a11 gene or transcript. In some examples, the RNAi is a shRNA, siRNA, or anti-sense RNA. In some embodiments, the modified PBMC includes a non-naturally occurring genetic modification of Slc16a11 that reduces an amount of functional MCT11. In some examples, the genetic modification is a point mutation, a partial deletion, a full deletion, or an insertion in a Slc16a11 gene, that reduces expression of Slc16a11 and/or reduces activity of MCT11.

In some embodiments, the modified PBMC is a T cell, for example, a CD8+ T cell or a CD3+ T cell. In some examples, the T cell is reactive to a tumor-specific antigen, for example, CD19, CD20, BCMA, MUC1, PSA, CEA, HER1, HER2, TRP-2, EpCAM, GPC3, mesothelin 1(MSLN), or EGFR. In further examples, the T cell is a tumor-infiltrating lymphocyte (TIL), and/or the T cell expresses a chimeric antigen receptor (CAR) or engineered T cell receptor (TCR). In some examples, the T cell is an exhausted T cell (including terminally exhausted T cells).

The disclosed modified PBMCs are useful, for example, to improve a cancer immunotherapy or to treat cancer or a tumor in vivo.

Also provided herein are methods of generating the modified PBMCs disclosed herein, for example, by introducing the agent that reduces Slc16a11 expression or the non-naturally occurring genetic modification that reduces functional MCT11 into a PBMC, thereby generating the modified PBMC with reduced expression of Slc16a11 and/or reduced activity of MCT11. In some examples, such methods further include selecting modified PBMCs having reduced expression of Slc16a11, reduced activity of MCT11, or both (such as purifying or isolating such cells away from cells not having reduced expression of Slc16a11, not having reduced activity of MCT11, or both). In some examples, such methods are performed ex vivo. In some examples, such selection methods are performed using flow cytometry, panning or magnetic separation. The disclosed methods in some examples further include introducing the selected modified PBMCs having reduced expression of Slc16a11, reduced activity of MCT11, or both, into a subject, such as a subject with a cancer to be treated with the selected modified PBMCs having reduced expression of Slc16a11, reduced activity of MCT11, or both.

Also provided herein are methods of treating cancer (or a tumor) or enhancing cancer immunotherapy by administering modified PBMCs disclosed herein to a subject having cancer (or a tumor) and/or receiving an immunotherapy, thereby treating the cancer (or tumor) or enhancing the immunotherapy. In some examples, the subject has or will receive a cancer immunotherapy, for example, a checkpoint inhibitor such as an anti-PD-1 or anti-PD-L1 monoclonal antibody therapy. In some examples, the subject is administered a small molecule inhibitor of MCT (e.g., MCT11) before, after, or substantially at the same time as the modified PBMCs.

The foregoing and other objects and features of the disclosure will become more apparent from the following detailed description, which proceeds with reference to the accompanying figures.

### BRIEF DESCRIPTION OF THE DRAWINGS

FIG. 1 shows typical phenotypes of “progenitor-like” or “terminally exhausted” T cells.

FIGS. 2A-2C show that MCT11 is upregulated in mouse and human exhausted T cells. FIG. 2A shows FACS sorting of LN CD8⁺ and TIL CD8⁺ cells for RNA-seq (left), and that MCT11 is upregulated in exhausted T cells (PD1^{hi}Tim3⁺) (right). FIG. 2B shows MCT11 staining of human PBMCs, PD1/TIM3⁻ and PD1/TIM3⁺ cells from melanoma (MEL) or head and neck cancer (HNSCC) patients. Human tumor biopsy samples were stained with antibodies to CD8, PD-1, Tim-3 and MCT11 and analyzed by flow cytometry. FIG. 2B depicts the staining of MCT11 as a function of progression to exhaustion (PD-1+Tim3+). FIG. 2C shows MCT11 surface expression on exhausted or non-exhausted tumor infiltrating lymphocytes (TILs) from MC38 (adenocarcinoma) or MEER (head and neck cancer) models in C56/BL6J mice.

FIGS. 3A-3B show Slc16a11 Transcripts Per Million bases (TPM) from RNA-seq of the indicated cell types. MCT11 is expressed (upregulated) on the surface of exhausted T cells, especially those that infiltrate tumors (TIL) (FIG. 3A). FIG. 3B was generated from publicly available data and confirms that Slc16a11 is specific to tumor-infiltrating exhausted T cells.

FIG. 4 shows that exhausted T cells specifically take up lactic acid. A schematic of the experimental design is shown at the top while the graph below shows lactic acid uptake after a 30 minute incubation with lactic acid for each indicated cell type.

FIG. 5 shows that MCT11-inhibited T cells (by treatment with shRNA specific for Slc16a11) retain anti-tumor function (top), while MCT11 overexpression (by treatment with a vector expressing Slc16a11) promotes T cell exhaustion in a mouse model (bottom).

FIGS. 6A-D show that mice carrying a conditional deletion of MCT11 (Slc16a11) had smaller tumor volume, and increased T cell infiltration and increased T cell function. FIG. 6A shows the experimental set-up. FIG. 6B shows tumor volume. FIG. 6C shows increased T cell infiltration (percent live CD8+ cells) and increased T cell function (as measured by cytokine production after restimulation) within the exhausted T cell compartment. FIG. 6D shows that an MCT11 mAb does not stain exhausted T cells (Texh) cells in mice bearing the conditional deletion of MCT11.

FIG. 7 shows that CRISPR/Cas9 mediated deletion of Slc16a11 in OT-1 OVA-specific T cells generated superior therapeutic cells (decreased tumor area) after a single dose in a mouse model.

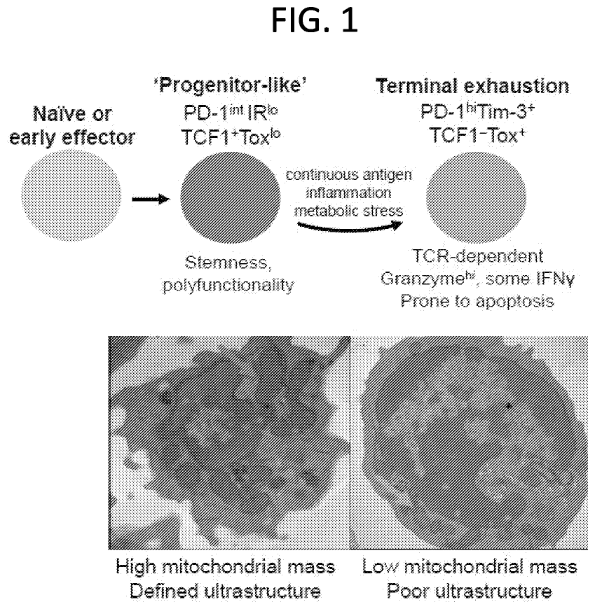

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. It is understood that when RNA is encoded as DNA, “U” is replaced with “T,” and conversely, when DNA is expressed as RNA, “T” is replaced with “U.”

The Sequence Listing is submitted as an XML file, “Sequence.xml,” created on Jul. 18, 2022, 16,384 bytes, which is incorporated by reference herein. In the accompanying sequence listing:

SEQ ID NO: 1: Exemplary amino acid sequence of human MCT11.
MPAPQRKHRRGGFSHRCFPTPQTAMTPQPAGPPDGGWGWVVAAAAFAINGLSYGLLRSL
GLAFPDLAEHFDRSAQDTAWISALALAVQQAASPVGSALSTRWGARPVVMVGGVLASLG
FVFSAFASDLLHLYLGLGLLAGFGWALVFAPALGTLSRYFSRRRVLAVGLALTGNGASSLL
LAPALQLLLDTFGWRGALLLLGAITLHLTPCGALLLPLVLPGDPPAPPRSPLAALGLSLFTR
RAFSIFALGTALVGGGYFVPYVHLAPHALDRGLGGYGAALVVAVAAMGDAGARLVCGW
LADQGWVPLPRLLAVFGALTGLGLWVVGLVPVVGGEESWGGPLLAAAVAYGLSAGSYA
PLVFGVLPGLVGVGGVVQATGLVMMLMSLGGLLGPPLSGFLRDETGDFTASFLLSGSLILS
GSFIYIGLPRALPSCGPASPPATPPPETGELLPAPQAVLLSPGGPGSTLDTTC
SEQ ID NO: 2: Exemplary nucleic acid sequence encoding human SLC16A11 mRNA.
CATTTCTCGCCGCTCCCCCCTCCCCCGGGCCTGGGGGTTGTGTGTGTACATAATTCAAT
CCCCGTGGGACTGGCGTCTGGCCTCACGCGCGGCTTGCCGGATTGGCTGTTTCAGCTCG
CCCCCGCCCTCCGTACACCCCGCGTCCCAGGTGGCTCAGGCCCCTGTGGTGATCTCTGT
TTACCGAGAGAGCCCGTCCAAGTTGGGCTCCATCGCTGCCCTCGCTCCCCTTCGGGGCC
CCCGCCCGCCTGGGAAGCAGAGAGAAAGCCGGGCCCAGCCCTTCCTCACCCTTCCCCT
CCCCGCACCGCCCGGAGAGGTCGGACGGCGATGACCCCCCAGCCCGCCGGACCCCCG
GATGGGGGCTGGGGCTGGGTGGTGGCGGCCGCAGCCTTCGCGATAAACGGGCTGTCCT
ACGGGCTGCTGCGCTCGCTGGGCCTTGCCTTCCCTGACCTTGCCGAGCACTTTGACCGA
AGCGCCCAGGACACTGCGTGGATCAGCGCCCTGGCCCTGGCCGTGCAGCAGGCAGCCA
GCCCCGTGGGCAGCGCCCTGAGCACGCGCTGGGGGGCCCGCCCCGTGGTGATGGTTGG
GGGCGTCCTCGCCTCGCTGGGCTTCGTCTTCTCGGCTTTCGCCAGCGATCTGCTGCATC
TCTACCTCGGCCTGGGCCTCCTCGCTGGCTTTGGTTGGGCCCTGGTGTTCGCCCCCGCC
CTAGGCACCCTCTCGCGTTACTTCTCCCGCCGTCGAGTCTTGGCGGTGGGGCTGGCGCT
CACCGGCAACGGGGCCTCCTCGCTGCTCCTGGCGCCCGCCTTGCAGCTTCTTCTCGATA
CTTTCGGCTGGCGGGGCGCTCTGCTCCTCCTCGGCGCGATCACCCTCCACCTCACCCCC
TGTGGCGCCCTGCTGCTACCCCTGGTCCTTCCTGGAGACCCCCCAGCCCCACCGCGTAG
TCCCCTAGCTGCCCTCGGCCTGAGTCTGTTCACACGCCGGGCCTTCTCAATCTTTGCTCT
AGGCACAGCCCTGGTTGGGGGCGGGTACTTCGTTCCTTACGTGCACTTGGCTCCCCACG
CTTTAGACCGGGGCCTGGGGGGATACGGAGCAGCGCTGGTGGTGGCCGTGGCTGCGAT
GGGGGATGCGGGCGCCCGGCTGGTCTGCGGGTGGCTGGCAGACCAAGGCTGGGTGCC
CCTCCCGCGGCTGCTGGCCGTATTCGGGGCTCTGACTGGGCTGGGGCTGTGGGTGGTG
GGGCTGGTGCCCGTGGTGGGCGGCGAAGAGAGCTGGGGGGGTCCCCTGCTGGCCGCG
GCTGTGGCCTATGGGCTGAGCGCGGGGAGTTACGCCCCGCTGGTTTTCGGTGTACTCCC
CGGGCTGGTGGGCGTCGGAGGTGTGGTGCAGGCCACAGGGCTGGTGATGATGCTGATG
AGCCTCGGGGGGCTCCTGGGCCCTCCCCTGTCAGGCTTCCTAAGGGATGAGACAGGAG
ACTTCACCGCCTCTTTCCTCCTGTCTGGTTCTTTGATCCTCTCCGGCAGCTTCATCTACA
TAGGGTTGCCCAGGGCGCTGCCCTCCTGTGGTCCAGCCTCCCCTCCAGCCACGCCTCCC
CCAGAGACGGGGGAGCTGCTTCCCGCTCCCCAGGCAGTCTTGCTGTCCCCAGGAGGCC
CTGGCTCCACTCTGGACACCACTTGTTGATTATTTTCTTGTTTGAGCCCCTCCCCCAATA
AAGAATTTTTATCGGGTTTTCCTGAAA
SEQ ID NO: 3: Exemplary guide RNA (gRNA) targeting sequence for human SLC16A11.
CGGGGGUCCGGCGGGCUGGG
SEQ ID NO: 4: Exemplary shRNA targeting human SLC16A11.
CCGGGCAGCUUCUUCUCGAUACUUUCUCGAGAAAGUAUCGAGAAGAAGCTGCUUUU
UG
SEQ ID NO: 5: Exemplary amino acid sequence of mouse MCT11.
MTPKPAGPPDGGWGWVVAAAAFAVNGLSYGLLRSLGLALPDLAEHFERSAQDTAWVSA
LALAVQQAASPVGSALSTRWGARPVVMVGGVLTSLGLVESAFARSLLHLYLGLGLLAGS
GWALVFAPALGTLSRYFSRRRVLAVGLALTGNGASSLLLAPALQFLLDTFGWRGALLLLG
AVTLHLTPCGALLRPLALSGDPLAPPRTPLAALGLGLFKRRAFSVFALGTALIGGGYFVPY
VHLGPHALDQGMGGYGAALVVAVAAVGDACARLASGWLADQGWVPLPRLLVVFGSLT
GLGVLAMGLVPTVGTEEGWGAPLLAAAGAYGLSAGSYAPLVFGVLPGLVGIGGVVQATG
LVMMLMSLGGLLGPPLSGFLRDKTGDFSASFLVCSSFILSGSFIYMGLPRALPSCRPASPPAT
PPPERGELLPVPQVSLLSAGGTGSIRDTTC
SEQ ID NO: 6: Exemplary nucleic acid sequence encoding mouse Slc16a11 mRNA.
GAGGCGGGGCGCGGCCACCAGGTACGCTCACTCCTCGCGGTTCCCCAACCAGGGCTGG
GGCTCGTGTGTGGATAATTCAATCCTCGTGGGACTCGGCGTCAGGCCTTAGCCAGGCC
TGGGTGGATTGGCTGTCTCTCTTTCCCCCGCCCTCGGTATTCACCCCCACCCCCACCCC
GTGTCCCAGGTGGCTTGGGCCCCTGTGGTGATCTCTGTTTACTGGAGAGAGCGGTCCA
AGTTGGGCTCCATCTCTGTGCTGGCTAGCTGCTCCGCAGAACCCTTTCTGCGGAAAGCG
CGGAGAAAATCCGGCCGAACCCACCTTCGCCCTTTCCCTACCCCCAACGCCGGGAGAG
GTCGGCAGACGGCGATGACCCCCAAGCCGGCCGGACCCCCGGACGGGGGCTGGGGCT
GGGTGGTGGCGGCCGCAGCATTCGCCGTGAACGGGCTCTCCTACGGGCTCTTACGCTC
CCTGGGCCTTGCCCTCCCCGACCTCGCGGAGCACTTTGAACGAAGCGCCCAGGACACT
GCGTGGGTCAGCGCCCTGGCCTTGGCCGTGCAGCAGGCAGCCAGCCCAGTGGGCAGCG
CCCTGAGCACTCGCTGGGGGGCACGCCCCGTGGTGATGGTTGGGGGAGTCCTAACCTC
GCTTGGCTTGGTCTTCTCGGCTTTCGCCCGAAGCCTCCTGCACCTCTACCTCGGCCTGG
GCCTCCTCGCTGGCTCTGGCTGGGCCCTGGTGTTTGCCCCAGCCCTGGGTACCCTCTCT
CGGTACTTCTCCCGCCGTCGGGTCTTGGCGGTAGGGTTGGCGCTCACCGGTAATGGGG
CATCCTCGCTGCTCCTGGCACCTGCCTTGCAGTTCCTCCTTGATACTTTCGGCTGGCGG
GGTGCCTTGCTCCTCCTTGGCGCTGTCACCCTTCACCTCACACCCTGTGGCGCCTTGCT
AAGACCTTTAGCTCTCTCTGGTGACCCGCTGGCCCCACCTCGCACCCCCCTAGCTGCCC
TTGGCCTAGGTCTGTTCAAGCGCCGGGCCTTTTCAGTCTTTGCTTTGGGCACCGCCTTG
ATCGGGGGCGGATACTTCGTCCCCTACGTTCATTTGGGTCCGCATGCTTTAGATCAAGG
CATGGGTGGTTATGGGGCAGCGTTAGTGGTGGCTGTCGCTGCAGTGGGAGATGCCTGT
GCCCGATTGGCCAGCGGATGGCTGGCAGACCAGGGCTGGGTGCCCCTTCCGAGGCTTC
TGGTGGTGTTTGGGTCTCTGACTGGGTTAGGGGTACTAGCAATGGGACTAGTGCCCAC
TGTGGGGACAGAGGAGGGTTGGGGGGCTCCTCTGCTGGCCGCTGCTGGGGCCTATGGG
CTGAGCGCTGGGAGTTATGCCCCACTGGTTTTCGGTGTGCTCCCGGGGCTGGTGGGCAT
TGGAGGTGTGGTGCAGGCCACAGGGCTGGTGATGATGCTGATGAGCCTCGGGGGACTC
CTGGGCCCTCCTCTGTCAGGCTTCCTAAGGGATAAGACAGGAGACTTCAGTGCCTCTTT
CCTGGTGTGCAGCTCTTTCATCCTCTCTGGCAGTTTCATCTACATGGGGCTGCCCAGAG
CCCTCCCCTCCTGCCGTCCAGCCTCACCTCCAGCAACCCCTCCACCAGAGAGAGGGGA
GCTGCTCCCAGTTCCACAAGTCTCCCTGCTTTCCGCAGGGGGTACTGGCTCCATCCGGG
ATACCACTTGTTGATCATTTTCTTGGTTGACCTCCTTCCCTAATAAAGAATTTTTATCTT
ACAAAAAAAAAAAAAAAAA
SEQ ID NO: 7: Exemplary guide RNA (gRNA) targeting sequence for mouse Slc16a11.
CGCCCCCUUCUAGGCCCAGU
SEQ ID NO: 8: Exemplary shRNA targeting mouse Slc16a11.
UGGCUUGGUCUUCUCGGCUUU

### DETAILED DESCRIPTION

Unless otherwise noted, technical terms are used according to conventional usage. Definitions of many common terms in molecular biology may be found in Krebs et al. (eds.), Lewin's genes XII, published by Jones & Bartlett Learning, 2017; The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

About: Unless context indicated otherwise, “about” refers to plus or minus 5% of a reference value. For example, “about” 100 refers to 95 to 105.

Administration: To provide or give a subject an agent, such as a modified PBMC, by any effective route. Administration can be local or systemic. Exemplary routes of administration include, but are not limited to, injection (such as subcutaneous, intramuscular, intradermal, intraperitoneal, intratumoral, intraprostatic, intrathecal, intraosseous, and intravenous), oral, sublingual, rectal, transdermal, intranasal, vaginal and inhalation routes. In some examples, modified PBMCs provided herein (such as T cells) are administered by intravenous injection.

Adoptive Cell Transfer (ACT) Therapy: A type of inmunotherapy in which a T cell that has been modified (e.g., modified to recognize a tumor antigen) and/or expanded in vitro (or ex vivo) is administered to a patient in need thereof. T cells for ACT therapy can be a patient's own T cells or T cells from a donor. ACT therapies include, for example, Chimeric Antigen Receptor T cell (CAR-T), Engineered T Cell Receptor (TCR), or Tumor-Infiltrating Lymphocyte (TIL) therapies. ACT therapy is also sometimes referred to as adoptive cell therapy, cellular adoptive immunotherapy, or T-cell transfer therapy.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

Cas9: An RNA-guided DNA endonuclease enzyme that that participates in the CRISPR-Cas immune defense against prokaryotic viruses. Cas9 has two active cutting sites (HNH and RuvC), one for each strand of the double helix. Thus, Cas9 proteins can be used to edit DNA in combination with an appropriate guide RNA. Cas9 sequences are publicly available. For example, GenBank® Accession Nos. nucleotides 796693 . . . 800799 of CP012045.1 and nucleotides 1100046 . . . 1104152 of CP014139.1 disclose exemplary Cas9 nucleic acids, and GenBank® Accession Nos. NP_269215.1, AMA70685.1, and AKP81606.1 disclose exemplary Cas9 proteins.

Catalytically inactive (deactivated or dead) Cas9 (dCas9) proteins, which have reduced or abolished endonuclease activity but still binds to dsDNA, are also encompassed by this disclosure. In some examples, a dCas9 includes one or more mutations in the RuvC and HNH nuclease domains, such as one or more of the following point mutations: D10A, E762A, D839A, H840A, N854A, N863A, and D986A. An exemplary dCas9 sequence includes both a D10A and H840A substitutions. In one example, the dCas9 protein has mutations D10A, H840A, D839A, and N863A (see, e.g., Esvelt et al., Nat. Meth. 10:1116-21, 2013). Exemplary dCas9 sequences are provided in GenBank® Accession Nos. AKA60242.1 and KR011748.1.

Cas13d: An RNA-guided RNA endonuclease enzyme that can cut or bind RNA. Cas13d proteins specifically recognize direct repeat (DR) sequences present in sgRNA having a particular secondary structure. Cas13d proteins include one or two HEPN domains. Native HEPN domains include the sequence RXXXXH, wherein X is any amino acid. A catalytically inactive, or “dead” Cas13d (dCas13d), which include mutated HEPN domain(s) and thus cannot cut RNA, but can process sgRNA, are also encompassed by this disclosure. dCas13d can be targeted to cis-elements of pre-mRNA to manipulate alternative splicing. Exemplary native and variant Cas13d protein sequences are provided in WO 2019/040664, U.S. Pat. Nos. 10,876,101 and 10,392,616.

In one example, a full length (non-truncated) Cas13d protein is between 870-1080 amino acids long. In one example, the Cas13d protein is derived from a genome sequence of a bacterium from the Order Clostridiales or a metagenomic sequence. In one example, the corresponding DR sequence of a Cas13d protein is located at the 5′ end of the spacer sequence in the molecule that includes the Cas13d gRNA. In one example, the DR sequence in the Cas13d sgRNA is truncated at the 5′ end relative to the DR sequence in the unprocessed Cas13d guide array transcript (such as truncated by at least 1 nt, at least 2 nt, at least 3 nt, at least 4 nt, at least 5 nt, such as 1-3 nt, 3-6 nt, 5-7 nt, or 5-10 nt). In one example, the DR sequence in the Cas13d gRNA is truncated by 5-7 nt at the 5′ end by the Cas13d protein. In one example, the Cas13d protein can cut a target RNA flanked at the 3′ end of the spacer-target duplex by any of a A, U, G or C ribonucleotide and flanked at the 5′ end by any of a A, U, G or C ribonucleotide.

Checkpoint Inhibitor (or Checkpoint Blockade): A therapeutic that targets a checkpoint protein. Checkpoint proteins help prevent over-active immune responses or autoimmunity, and can sometimes limit T cell ability to eliminate cancerous cells. When checkpoints are blocked (e.g., PD-1 blockade) T cells can better target and kill cancerous cells. Examples of checkpoint proteins found on T cells or cancerous cells include PD-1/PD-L1/PD-L2, and CTLA-4/B7-1/B7-2.

Exemplary checkpoint inhibitors include ipilimumab (Yervoy®), nivolumab (Opdivo@), pembrolizumab (Keytruda®), atezolizumab (Tencentriq@), avelumab (Bavencio@), durvalumab (Imfinzi@), cemiplimab (Libtayo®), palbociclib (Ibrance@), ribociclib (Kisquali@), pidilizumab, avelumab, and abemaciclib (Verzenio@). Further examples are provided in Qiu et al., Journal of the European Society for Therapeutic Radiology and Oncology, 126(3):450-464, 2018; Visconti et al., J Exp Clin Cancer Res. 35(1): 153, 2016; and Mills et al. Cancer Res. 77(23): 6489-6498, 2017.

Chimeric antigen receptor (CAR): Artificial, engineered T cell receptors, which graft an arbitrary specificity onto an immune effector cell. Typically, these receptors are used to graft the specificity of a monoclonal antibody onto a T cell; with transfer of their coding sequence facilitated by vectors. Thus, a CAR that “specifically binds” or is “specific” for an antigen is a CAR that binds the antigen with high affinity and does not significantly bind other unrelated antigens. Using adoptive cell transfer, CARs can be useful to treat cancer. For example, T cells (obtained from the patient or from a donor) are modified such that they express receptors specific to the patient's particular cancer. The modified T cells, which can then recognize and kill the cancer cells, are introduced into the patient. In some examples, the modified PBMC disclosed herein express a CAR.

First generation CARs typically included the intracellular domain from the CD3 ζ- chain, which is the primary transmitter of signals from endogenous TCRs. Second generation CARs added intracellular signaling domains from various costimulatory protein receptors (e.g., CD28, 41BB, ICOS) to the cytoplasmic tail of the CAR to provide additional signals to the T cell. Third generation CARs combine multiple signaling domains, such as CD3z-CD28-41BB or CD3z-CD28-OX40, to augment potency. A multispecific CAR is a single CAR molecule comprised of at least two antigen-binding domains (such as scFvs and/or single-domain antibodies) that each bind a different antigen or a different epitope on the same antigen (see, for example, US 2018/0230225). For example, a bispecific CAR refers to a single CAR molecule having two antigen-binding domains that each bind a different antigen. A bicistronic CAR refers to two complete CAR molecules, each containing an antigen-binding moiety that binds a different antigen. In some cases, a bicistronic CAR construct expresses two complete CAR molecules that are linked by a cleavage linker. T cells expressing a bispecific or bicistronic CAR can bind cells that express both of the antigens to which the binding moieties are directed (see, for example, Qin et al., Blood 130:810, 2017; and WO/2018/213337). Any of these CARs can be used with the methods described herein.

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, and 10 out of 10 being 50%, 60%, 70%, 80%, 90%, and 100% complementary, respectively).

In some embodiments, a disclosed nucleic acid molecule (such as a disclosed gRNA or RNAi) hybridizes to a target nucleic acid, thus the nucleic acid molecule is complementary to the target sequence. For example, in some examples, a RNAi or gRNA specific for Slc16a11 gene or transcript is at least 80%, at least 85%, at least 90%, at least 91%, at least 92%, at least 93%, at least 94%, at least 95%, at least 96%, at least 97%, at least 98% or at least 99% complementary to a unique portion of a target gene, such as Slc16a11. In some examples, the target sequence is at least 10 contiguous nucleotides, for example, at least 12, at least 15, at least 20, at least 25, at least 30, at least 35, or at least 40 contiguous nucleotides. In further examples, the target sequence is 10-50 contiguous nucleotides, for example, 12-40, 12-30, 12-20, 12-15, 15-30, 15-20, 20-30, or 20-40 contiguous nucleotides. In some examples, the targeting sequence is at least 80%, at least 85%, at least 90%, at least 91%, at least 92%, at least 93%, at least 94%, at least 95%, at least 96%, at least 97%, at least 98% or at least 99% complementary to a sequence about 20 nucleotides long in a Slc16a11 gene or transcript.

Control: A reference standard. In some embodiments, the control is a negative control. In other embodiments, the control is a positive control. In some examples, a suitable control is a historical control or standard reference value or range of values (such as a previously tested control sample, such as a group of patients diagnosed with a disease or condition, for example cancer, that have a known prognosis or outcome, or a group of samples that represent baseline or normal values). In some examples, the control may be a subject not receiving treatment with an agent (e.g., the disclosed modified PBMCs) or receiving an alternative treatment, or a baseline reading of the subject prior to treatment with an agent.

A difference between a test sample and a control can be an increase or conversely a decrease. The difference can be a qualitative difference or a quantitative difference, for example a statistically significant difference. In some examples, a difference is an increase relative to a control, for example, an increase of at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%. In other examples, a difference is a decrease relative to a control, for example, a decrease of at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, or at least about 100%.

CRISPR/Cas9 system: A prokaryotic immune system that confers resistance to foreign genetic elements, such as plasmids and phages, and provides a form of acquired immunity. In the endogenous system, a trans-activating crRNA (tracrRNA) hybridizes with the repeat sequence of another RNA molecule known as CRISPR RNA (crRNA) to form a unique dual-RNA hybrid structure that binds Cas9 endonuclease, forming a ribonucleoprotein (RNP) complex. The crRNA contains a targeting sequence complementary to a target gene, which guides the CRISPR/Cas9 RNP complex to the target. Cas9 then induces a double stranded DNA break in the target gene. The CRISPR/Cas9 system can be used to decrease gene expression, for example, by targeting and inducing double-stranded DNA breaks in a target gene, such as Slc16a11. Similarly, a CRISPR/Cas13 system can be used to cut RNA.

Effective amount: The amount of an agent (such as the modified PBMC, RNAi, gRNA, or other composition disclosed herein) that is sufficient to effect beneficial or desired results. An effective amount (also referred to as a therapeutically effective amount) may vary depending upon one or more of: the subject and disease condition being treated, the weight and age of the subject, the severity of the disease condition, the manner of administration and the like, which can readily be determined by one of ordinary skill in the art. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one embodiment, an “effective amount” of a therapeutic agent (e.g., a modified PBMC disclosed herein) is an amount sufficient to reduce the volume/size of a tumor, the weight of a tumor, the number of metastases, reduce the volume/size of a metastasis, the weight of a metastasis, or combinations thereof, for example by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99% (as compared to a suitable control, such as no administration of the therapeutic agent). In one embodiment, an “effective amount” of a therapeutic agent (e.g., a gRNA or siRNA disclosed herein) is an amount sufficient to reduce activity or expression of a target (e.g., MCT11), for example by at least 5%, at least 10%, at least 15%, at least 20%, at least 25%, at least 30%, at least 40%, at least 50%, at least 60%, at least 70%, at least 75%, at least 80%, at least 90%, at least 95%, at least 98%, or even 100% (as compared to a suitable control, such as expression or activity prior to administering the therapeutic agent). In some examples, combinations of these effects are achieved.

Guide RNA (gRNA): An RNA component of a CRISPR/Cas system that targets the CRISPR/Cas ribonucleoprotein (RNP) complex to a target nucleic acid sequence, such as a target DNA (e.g., genomic sequence) or target RNA sequence. gRNA molecules include (1) a portion with sequence complementarity to the target nucleic acid (such as at least 80%, at least 90%, at least 95%, or 100% sequence complementarity), and (2) a portion with secondary structure that binds to the Cas nuclease. Such portions can be part of the same molecule (e.g., sgRNA: a synthetic chimera that combines a crRNA and tracrRNA into a single RNA transcript), or divided over two or more separate molecules (e.g., 2 part gRNA wherein the crRNA and tracrRNA are separate RNA transcripts). For simplicity, both types of molecules are referred to herein as gRNA.

Many techniques for genome editing using the CRISPR/Cas system have been described. In brief, gRNA directs a Cas DNA nuclease (such Cas9) to a target gene (DNA). Cas9 then introduces a double stranded break at the target site. Disruptive mutations can be introduced through non-homologous end joining of the cut DNA. Cas9 can also be used to delete larger DNA fragments, for example, by using two gRNAs targeting separate sites, thus causing a deletion of the intervening sequence between the two cut sites. A DNA template with homology to the target site can also be added to introduce insertions using homology directed DNA repair mechanisms.

In RNA editing, the gRNA directs a Cas RNA nuclease (such Cas13d) to a target RNA. In one such example, the gRNA includes from 5′ to 3′ (1) a crRNA containing a direct repeat (DR) region and (2) a spacer, for example for Cas13a, Cas13c, and Cas 13d nucleases. In one example includes about 36nt of DR followed by about 28-32nt of spacer sequence. In another such example, the gRNA includes from 5′ to 3′ (1) a spacer and (2) a crRNA containing a DR region, for example for Cas13b nuclease. In some examples, the gRNA is processed (truncated/modified) by a Cas RNA nuclease or other RNases into the shorter “mature” form. The DR is the constant portion of the sgRNA, containing secondary structure which facilitates interaction between the Cas RNA nuclease protein and the gRNA. The spacer portion is the variable portion of the gRNA, and includes a sequence designed to hybridize to a target RNA sequence (and in some examples edit the target RNA sequence). In some examples, the full length spacer is about 28-32nt (such as 30-32 nt) long while the mature (processed) spacer is about 14-30nt.

The targeting portion of the gRNA can be modified to facilitate targeting of any DNA or RNA sequence of interest. (See CRISPR-Cas9 Structures and Mechanisms. Fuguo Jiang and Jennifer A. Doudna, Annual Review of Biophysics, 46:1, 505-529 (2017)). A gRNA that is “specific” for a target has sufficient complementarity to the target sequence that it binds the target and does not significantly hybridize with other unrelated sequences. The targeting sequence of the gRNA is typically about 20 nucleotides, for example, about 15, about 16, about 17, about 18, about 19, about 20, about 21, about 22, about 23, about 24, or about 25 nucleotides. The degree of complementarity between a targeting sequence and its corresponding target sequence, when optimally aligned using a suitable alignment algorithm, is about or more than about 50%, about 60%, about 75%, about 80%, about 85%, about 90%, about 95%, about 97.5%, about 98%, about 99%, or more. In some embodiments, the degree of complementarity is 100%. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some examples, the targeting sequence is at least 80%, at least 85%, at least 90%, at least 91%, at least 92%, at least 93%, at least 94%, at least 95%, at least 96%, at least 97%, at least 98% or at least 99% complementary to a contiguous amino acid sequence about 20 nucleotides long in a Slc16a11 gene or transcript.

Immunotherapy: A therapy that uses an agent to stimulate or suppress the immune system to treat a disease, such as cancer. Some examples of cancer immunotherapy include immune checkpoint inhibitors, adoptive cell transfer (ACT) immunotherapy, antibodies, vaccines, and immune system modulators. Specific, non-limiting examples include nivolumab, pembrolizmab, pidilizumab, atezolizumab, durvalumab, avelumab, and ipilimumab.

Increase or Decrease: A positive or negative change, respectively, in quantity from a control value (such as a value representing no therapeutic agent). An increase is a positive change, such as an increase at least 25%, at least 50%, at least 100%, at least 200%, at least 300%, at least 400% or at least 500%, as compared to the control value. A decrease is a negative change, such as a decrease of at least 10%, at least 20%, at least 30%, at least 40%, at least 50%, at least 60%, at least 70%, at least 75%, at least 80%, at least 85%, at least 90%, at least 95%, at least 98%, at least 99%, or at least 100% decrease as compared to a control value. In some examples, the increase or decrease is statistically significant relative to a suitable control.

An agent (e.g., the RNAi or a gRNA specific for Slc16a11 disclosed herein) that decreases expression or activity of a gene (e.g., Slc16a11) or gene product (e.g., MCT11) is a compound that reduces the level of the mRNA or a functional product encoded by the gene in a cell or tissue (e.g., a PBMC), or reduces (including eliminates or inhibits) one or more activities of the gene product. In some embodiments, expression of Slc16a11 is reduced at least 10%, at least 25%, at least 50%, at least 75%, at least 90%, at least 95%, at least 99%, at least 99.9%, or even 100% relative to a control, such as an untreated subject or cells. Conversely, an agent that increases expression or activity of a gene or gene product is a compound that increases the level of the mRNA or protein product encoded by the gene in a cell or tissue, or increases one or more activities of the gene product.

In some embodiments, an agent (e.g., the RNAi or a gRNA specific for Slc16a11 disclosed herein) or non-naturally occurring genetic modification can increase or decrease an activity of a PBMC (e.g., a T cell) when it is present in the PBMC. For example, in some embodiments the PBMC is a T cell and the agent (e.g., the RNAi or a gRNA specific for Slc16a11 disclosed herein) or genetic modification (a point mutation, a partial deletion, full deletion, or insertion that reduces expression of Slc16a11, as disclosed herein) reduces T cell exhaustion, for example, by at least 10%, at least 20%, at least 30%, at least 40%, at least 50%, at least 60%, at least 70%, at least 80%, at least 90%, at least 95%, or 100% relative to a suitable control (e.g., measurements prior to treatment or comparison to an untreated control group). A decrease in T cell exhaustion can be measured, for example, by a decrease in lactic acid uptake, a decrease in expression of PD-1 or Tim3, an increase in cytokine production (e.g., INF-γ, TNFα, or IL-2), an increase in cytotoxic activity (e.g., increased tumor specific targeting or killing), or by measuring another indicator of T cell effector activity, relative to a suitable control. In some examples, combinations of these effects are achieved.

In some embodiments the agent (e.g., the RNAi or a gRNA specific for Slc16a11 disclosed herein) or genetic modification (a point mutation, a partial deletion, full deletion, or insertion that reduces expression of Slc16a11, as disclosed herein) increases the activity or function of the PBMC. For example, in some examples the PBMC is a T cell and the agent can increase the activity or effector function of a T cell by at least 10%, at least 25%, at least 50%, at least 75%, at least 90%, at least 95%, at least 100%, at least 200%, at least 300%, at least 400%, or at least 500% relative to a suitable control (e.g., measurements prior to treatment or comparison to an untreated control group). An increase in effector function of a T cell can be measured, for example, by a decrease in lactic acid uptake, a decrease in expression of PD-1 or Tim3, an increase in cytokine production (e.g., INF-γ, TNFα, or IL-2), an increase in cell proliferation (in vitro or in vivo expansion), or an increase in cytotoxic activity (e.g., increased tumor specific targeting or killing), or by another indicator of effector function, relative to a suitable control. In some examples, combinations of these effects are achieved.

Isolated: An “isolated” biological component (e.g., a cell, PBMC, nucleic acid, protein) has been substantially separated, produced apart from, or purified away from other biological components in the cell or tissue of an organism in which the component occurs, such as other cells (e.g., RBCs), chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. For example, PBMCs or TILs isolated from patient blood, tumor, or other sample, are at least 50% pure, such as at least 60%, such as at least 75%, at least 80%, at least 90%, at least 95%, at least 98%, at least 99%, or more, pure.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (for example, a promoter that drives expression of the heterologous nucleic acid sequence encoding the siRNA or gRNA disclosed herein). Generally, operably linked DNA sequences are contiguous and, where necessary to join two protein coding regions, are in the same reading frame.

Programmed cell death protein 1 (PD-1): A cell surface receptor that belongs to the immunoglobulin superfamily and is expressed on T cells and pro-B cells. PD-1 binds two ligands, PD-L1 and PD-L2. The human form is a 268 amino acid type 1 transmembrane protein. PD-1 is an inhibitory receptor that suppresses T cell activity and mediates T-cell exhaustion. PD-1 sequences are publicly available, for example from the GenBank® sequence database (e.g., Accession Nos. NP_005009.2 (mature peptide is aa 21-288), CAA48113.1, NP_001301026.1 (mature peptide is aa 25-288), and CAA48113.1 (mature peptide is aa 21-288) provide exemplary PD-1 protein sequences, while Accession Nos. L27440.1, NM_005018.2, X67914.1, AB898677.1 and EU295528.2 provide exemplary PD-1 nucleic acid sequences).

Pharmaceutically acceptable carriers: The pharmaceutically acceptable carriers useful in this invention are conventional. Remington's Pharmaceutical Sciences, 23rd Edition, Academic Press, Elsevier, (2020), describes compositions and formulations suitable for pharmaceutical delivery of a therapeutic agent, such as modified PBMCs disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, 5% human serum albumin, glycerol, or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. Supplementary active compounds can also be incorporated into the compositions.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Examples of promoters include, but are not limited to the SV40 promoter, the CMV enhancer-promoter, the CMV enhancer/β-actin promoter, EF1a promoter, or PGK promoter. In one example, expression of a gRNA is driven by a polymerase III promoter, such as U6 or H1, such as human or mouse U6 or H1 promoter. Both constitutive and inducible promoters are included (see e.g., Bitter et al., Methods in Enzymology 153:516-544, 1987). Also included are those promoter elements that are sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Prevent: Preventing a condition refers to reducing, delaying, or inhibiting the full development of a condition, for example preventing, reducing, or slowing the progression of a T cell to an exhausted T cell. In one example an agent that reduces Slc16a11 expression, or a non-naturally occurring genetic modification that reduces an amount of functional MCT11, when present in a PBMC, such a T cell, such as a CAR or TCR, prevents or reduces the likelihood that the cell will become exhausted (e.g., prevents or reduces the likelihood a T cell will overexpress programmed cell death 1 (PD1^{hi}) and/or become positive for T cell immunoglobulin and mucin domain-containing protein 3 (TIM3⁺), or may slow the progression of the cell to an exhausted state. In some examples, the disclosed modified PBMCs, such as modified T cells, do not become exhausted. In some examples, the disclosed modified PBMCs, such as modified T cells, show a reduction in exhaustion, such as a reduction of least 10%, at least 25%, at least 50%, at least 75%, at least 90%, at least 95%, at least 99%, or at least 99.9%, relative to an unmodified PBMC or T cell. In some examples, the disclosed modified PBMCs, such as modified T cells, show a slower progression to exhaustion, such as an increase in the number of days to exhaustion of least 10%, at least 25%, at least 50%, at least 75%, at least 90%, at least 95%, at least 99%, slower relative to an unmodified PBMC or T cell.

RNA interference or interfering RNA (RNAi): A cellular process that inhibits expression of genes, including cellular and viral genes. RNAi is a form of antisense-mediated gene silencing involving the introduction of double stranded RNA-like oligonucleotides leading to the sequence-specific reduction of RNA transcripts. RNA molecules that inhibit gene expression through the RNAi pathway can include siRNAs, miRNAs, gRNAs, and shRNAs. In one example, an RNAi is specific for Slc16a11, and can specifically hybridize to a Slc16a11 nucleic acid molecule.

Sequence identity: The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are. Homologs of a polypeptide (or nucleotide sequence) will possess a relatively high degree of sequence identity when aligned using standard methods.

Methods of alignment of sequences for comparison have been described. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Short hairpin RNA (shRNA): A sequence of RNA that makes a tight hairpin turn and can be used to silence gene expression via the RNAi pathway. The shRNA hairpin structure is cleaved by cellular machinery into siRNA. A shRNA that is “specific” for a target sequence (such as Slc16a11) has sufficient complementarity to the target sequence that it binds the target and does not significantly hybridize with other unrelated sequences.

Solute carrier family 16 member 11 (Slc16a11) and Monocarboxylate transporter 11 (MCT11): Slc16a11 is a gene encoding MCT11 protein. MCT11 is a recently characterized transporter protein that may transport monocarboxylates, such as lactic acid. SEQ ID NO: 1 discloses an exemplary amino acid sequence of MCT11. SEQ ID NO: 2 discloses an exemplary nucleic acid sequence of Slc16a11. Sequences for MCT11/Slc16a11 are publicly available, for example, see UniProt Accession No. Q8NCK7, GenBank Accession Nos: KJ900348.1, NM_153081.3, NM_153357.3, and NM_001370549.1 provide exemplary Slc16a11 nucleic acid sequences, and GenBank Accession Nos: NP_001357478.1, NP_694721.2 and NP_001099267.2 provide exemplary MCT11 protein sequences. In one example, a MCT11 protein has at least 80%, at least 85%, at least 90%, at least 91%, at least 92%, at least 95%, at least 96%, at least 97%, at least 98%, at least 99%, or 100% sequence identity to SEQ ID NO: 1, to GenBank Accession No. NP_001357478.1, NP 694721.2 or NP_001099267.2, or the sequence of UniProt Accession No. Q8NCK7. In one example, a Slc16a11 nucleic acid molecule has at least 80%, at least 85%, at least 90%, at least 91%, at least 92%, at least 95%, at least 96%, at least 97%, at least 98%, at least 99%, or 100% sequence identity to SEQ ID NO: 2, to GenBank Accession No. KJ900348.1, NM_153081.3, NM_153357.3, or NM_001370549.1, or the sequence of UniProt Accession No. Q8NCK7.

Small interfering RNA (siRNA): A double-stranded nucleic acid molecule that modulates gene expression through the RNAi pathway. siRNA molecules are generally 15 to 40 nucleotides in length, such as 20-30 or 20-25 nucleotides in length, with 0 to 5 (such as 2)-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs.” A siRNA that is “specific” for a target sequence (such as Slc16a11) has sufficient complementarity to the target sequence that it binds the target and does not significantly hybridize with other unrelated sequences.

Small molecule inhibitor: A molecule, typically with a molecular weight less than about 1000 Daltons, or in some embodiments, less than about 500 Daltons, wherein the molecule is capable of modulating, to some measurable extent, an activity of a target molecule (e.g., MCT11). In some examples, the small molecule inhibitor is a MCT inhibitor (e.g., 7ACC1, AR-C155858, UK 5099, SR 13800, CHC, AR-C 141990 hydrochloride, AZD3965, or BAY8002).

Subject: A vertebrate, such as a mammal, for example a human. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dog, cat, horse, or cow. In some examples, the subject has cancer (or a tumor), that can be treated using the modified PBMCs disclosed herein. In some examples, the subject is a laboratory animal/organism, such as a mouse, rabbit, or rat.

T cell agonists: an immunotherapy that activates T-cells to promote anti-tumor function. Non-limiting examples include urelumab and utomilumab.

T cells: A white blood cell (lymphocyte) that is an important mediator of the immune response. T cells include, but are not limited to, CD3⁺ T cells, CD4⁺ T cells and CD8⁺ T cells. A CD4⁺ T cell is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In some examples, a CD8⁺ T cell is a cytotoxic T lymphocyte (CTL). CD3⁺ T cells carry the “cluster of differentiation 3” (CD3) marker, a multimeric protein complex historically known as the T3 complex.

Activated T cells can be detected by an increase in cell proliferation and/or expression of or secretion of one or more cytokines (such as IL-2, IL-4, IL-6, IFN-γ, or TNFα). Activation of CD8⁺ T cells can also be detected by an increase in cytolytic activity in response to an antigen. “Exhausted T cells” are dysfunctional T cells (hyporesponsive) commonly found in cancer environments. T cell exhaustion is characterized by a progressive loss of effector function (for example, loss of IL-2, TNF-α, and IFN-γ production) and sustained expression of inhibitory receptors such as PD-1, T cell immunoglobulin domain and mucin domain-containing protein 3 (Tim-3), CTLA-4, lymphocyte-activation gene 3 (LAG-3), and CD160. In some examples, the exhausted T cell is a CD3⁺ T cell or CD8⁺ T cell. In some examples, the exhausted T cell is a terminally exhausted T cell (a terminally differentiated T cell that is exhausted). Terminally exhausted T cells express Tim3 and have high and persistent expression of PD-1 relative to other T cells (Tim3⁺ PD-1^{hi} T cells). T cells that are Tim3⁺ and/or PD-1^{hi} can be determined by FACs analysis, for example, by FACs analysis of a population of T cells. In some examples, terminally exhausted T cells express MCT11. A possible cause of T cell exhaustion is chronic activation or prolonged antigen stimulation. In some examples, the modified PBMC is an exhausted T cell (including a terminally exhausted T cell).

A “Therapeutic T Cell” is a T cell that is used for therapy, such as immunotherapy (e.g., cancer immunotherapy). Therapeutic T cells are administered to a subject for treatment of a particular disease, for example, cancer or an immune disease. In some examples, the therapeutic T cell recognizes and kill target cells, for example, cancerous cells, thereby treating a disease, such as cancer. Therapeutic T cells may be autologous or allogeneic to the subject. In some examples, the therapeutic T cell is a T cell to be used for Adoptive Cell Transfer (ACT) immunotherapy. In further examples, the therapeutic T cell expresses a Chimeric Antigen Receptor (CAR) or Engineered T Cell Receptor (TCR), and/or is a Tumor-Infiltrating Lymphocyte (TIL).

T cell receptor (TCR): A receptor found on the surface of T lymphocytes (or T cells) responsible for recognizing fragments of antigen as peptides bound to major histocompatibility complex (MHC) molecules. The TCR is composed of two different protein chains. In humans, in 95% of T cells the TCR consists of an alpha (α) and beta (β) chain, whereas in 5% of T cells the TCR consists of gamma and delta (γ/δ) chains. This ratio changes during ontogeny and in diseased states as well as in different species. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors. In one example, a TCR is a recombinant TCR, such as one used in TCR-engineered T cells for ACT therapy.

Therapeutic agent: Refers to one or more molecules or compounds that confer some beneficial effect upon administration to a subject. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

Transformed: A transformed cell is a cell (such as a PBMC, such as a T cell) into which a nucleic acid molecule has been introduced by molecular biology techniques. As used herein, the term transformed and the like (e.g., transformation, transfection, transduction, etc.) encompass all techniques by which a nucleic acid molecule might be introduced into such a cell, including viral vectors, plasmid vectors, nucleic acid-protein complexes (e.g., ribonucleoprotein), or naked nucleic acids (e.g., oligonucleotides).

Exemplary methods of transformation include chemical methods (e.g., calcium-phosphate transfection), physical methods (e.g., electroporation, microinjection, particle bombardment), fusion (e.g., liposomes), lipofection, nucleofection, receptor-mediated endocytosis (e.g., DNA-protein complexes, viral envelope/capsid-DNA complexes), particle gun accelerator (gene gun), and by biological infection by viruses such as recombinant viruses (Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, blood and other clinical tests, and the like. In some examples, treatment with the disclosed methods results in a decrease in the number, volume, and/or weight of a tumor and/or metastases.

Tumor-Infiltrating Lymphocyte (TIL): lymphocytes that invade tumor tissue. For example, T cells found within a tumor sample. In ACT therapy, TIL therapy generally involves isolating TILs from a patient tumor, activating and expanding the TILs in culture, and then re-infusing into the patient. In some examples, the modified PBMC disclosed herein is a TIL.

Tumor, neoplasia, or malignancy: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be treated using the disclosed modified PBMCs include solid tumors, such as breast carcinomas (e.g. lobular and duct carcinomas, such as a triple negative breast cancer), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma, and lymphatic tumors (including B-cell and T-cell malignant lymphoma). In one example, the tumor is a melanoma. In one example, the tumor is a head and neck squamous cell carcinoma (HNSCC), such as an HPV-positive HNSCC.

The disclosed modified PBMCs can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

Vector: A nucleic acid molecule that can be introduced into a host cell (for example, by transfection or transformation), thereby producing a transformed host cell (such as a transformed PBMC). Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors (such as AVV) are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function.

### Overview

Immunotherapy has changed the treatment paradigm in cancer, most notably the use of blockade of checkpoint receptors as well as engineered, tumor-specific T cells for therapy. However, a potential factor limiting efficacy of these modalities is the development of T cell exhaustion, an alternative differentiation fate of T cells to a more dysfunctional state. Exhaustion limits the capacity of T cells to respond to immunotherapy. Exhausted T cells also have a distinct metabolic profile limiting their function. For example, they compete poorly for glucose and repress the generation of new mitochondria. However, these cells persist in the tumor microenvironment, thus certain metabolic pathways or metabolites sustaining these cells may hinder anti-tumor function.

Here, it is shown that terminally exhausted T cells specifically upregulate MCT11 expression. Terminally exhausted T cells can also specifically take up monocarboxylates, such as lactic acid. This indicates that the MCT11 transporter has a role in providing nutrient flux to terminally exhausted T cells. Deletion of MCT11 activity in tumor-specific T cells transferred into tumor-bearing mice resulted in increased T cell function and decreased exhaustion. Further, overexpression of MCT11 in tumor-specific T cells accelerated development of the exhausted, dysfunctional phenotype. Thus, without being bound to any particular theory, MCT11 likely supports uptake of lactic acid which may limit the effector function of exhausted T cells. Thus, reducing or eliminating MCT11 function or expression on T cells can be used to increase effector activity and/or decrease T cell exhaustion, for example prevent exhaustion of a T cell used in cancer therapy. Thus, in some examples, the disclosed modified T cells with reduced or eliminated MCT11 expression and/or activity are generated ex vivo, preventing or slowing the T cells ability to become exhausted, wherein the modified cells can be administered to a subject with cancer for immunotherapy. The use of PBMCs, such as therapeutic T cells, in such therapies, can provide a more robust immune response and better cancer treatment.

### I. RNAi and gRNA

Disclosed herein are interfering RNA (RNAi) or guide nucleic acid (gRNA) specific for Slc16a11 (SLC16A11). The RNAi or gRNA targets a Slc16a11 nucleic acid molecule, such as a gene or transcript, to reduce expression of Slc16a11. In some examples, the RNAi or gRNA are introduced into a cell, for example, a PBMC, T cell, or exhausted T cell (including a terminally exhausted T cell). In some examples, the RNAi or gRNA molecules are directly introduced into the cell, for example, as oligonucleotides. In some examples, RNAi of gRNA molecules are expressed from a vector that is introduced into the cell. In examples where a guide RNA is expressed (e.g., from an expression cassette or vector) the guide RNA may be encoded as DNA.

In some embodiments, an RNAi specific for a Slc16a11 gene or transcript is used to reduce or inhibit expression of Slc16a11. The specificity of the RNAi for Slc16a11 allows hybridization of the RNAi molecule to Slc16a11 DNA or RNA, thereby reducing or inhibiting Slc16a11 expression. RNAi generically refers to a cellular process that inhibits expression of genes by inhibiting transcription and/or translation. Molecules that inhibit gene expression through the RNAi pathway include siRNAs, miRNAs, antisense RNAs, and shRNAs. In some examples, the RNAi specific for Slc16a11 includes a sequence at least 70%, at least 80%, at least 90%, at least 95%, at least 99%, or 100% complementary to a unique (e.g., not found elsewhere in the genome of the cell or organism into which the RNAi is introduced) contiguous portion of a Slc16a11 gene or transcript (such as a portion of SEQ ID NO: 2). In some examples, the RNAi specific for Slc16a11 consists of a sequence at least 90% complementary (e.g., at least 91%, 92%, 93%, 94%, 95%, 96%, 97%, 98% 99% or 100% complementary) to a unique contiguous portion of Slc16a11 gene or transcript (such as a portion of SEQ ID NO: 2).

In a specific, non-limiting example, the RNAi is a shRNA specific for a Slc16a11 gene or transcript. Methods of designing shRNA have been described, for example, see Moore et al. (2010) Short Hairpin RNA (shRNA): Design, Delivery, and Assessment of Gene Knockdown, Methods Mol. Biol., 629:141-158, herein incorporated by reference in its entirety. In some examples, the shRNA is specific to a unique contiguous portion of a sequence with at least 70%, at least 80%, at least 90%, at least 95%, at least 98%, at least 99%, or 100% sequence identity to SEQ ID NO: 2. In some examples, the shRNA is specific to a unique contiguous portion of a sequence with at least 90% sequence identity to SEQ ID NO: 2.

In some examples, the shRNA includes a targeting sequence with at least 70%, at least 80%, at least 90%, at least 95%, at least 98%, at least 99%, or 100% sequence identity to nucleotides 5-25 of SEQ ID NO: 4. In some examples, the shRNA includes a targeting sequence with at least 95% sequence identity to nucleotides 5-25 of SEQ ID NO: 4. In some examples, the shRNA has at least 70%, at least 80%, at least 90%, at least 95%, at least 90%, at least 99%, or 100% sequence identity to SEQ ID NO: 4. For example, the shRNA can have at least 95% sequence identity to SEQ ID NO: 4. In some examples, the shRNA includes or consists of SEQ ID NO: 4.

In other examples, the RNAi is a siRNA specific for Slc16a11 gene or transcript, for example the siRNA is specific for a sequence with at least 70%, at least 80%, at least 90%, at least 95%, at least 98%, at least 99%, or 100% sequence identity to a unique contiguous portion of SEQ ID NO: 2. For example, the siRNA can be specific to a sequence with at least 95% sequence identity to a unique contiguous portion of SEQ ID NO: 2.

In some embodiments, a gRNA specific for a Slc16a11 gene or transcript is used to reduce or inhibit expression of Slc16a11. For example, CRISPR/Cas methods can be used with a gRNA specific for a Slc16a11 gene or transcript to reduce or inhibit expression of Slc16a11. The specificity of the gRNA for Slc16a11, in combination with a Cas nuclease or dead nuclease (such as Cas9, dCas9, dCas13d or Cas13d) allows hybridization of the gRNA molecule to Slc16a11 DNA or RNA, thereby editing the Slc16a11 (for example mutating it, such as knocking it down or knocking it out) to reduce or inhibit its expression. In some examples, the Cas nuclease (or a dead Cas nuclease) sequence is codon optimized for expression in a host cell. In some examples, gRNA molecules and Cas nucleases are expressed from a vector introduced into a host cell (e.g., PBMC, T cell, or exhausted T cell). In some examples, an RNP complex containing gRNA molecules and Cas nucleases are introduced into a cell (e.g., PBMC, T cell, or exhausted T cell). In some examples, the gRNAs are introduced into a cell, for example, as oligonucleotides.

In some examples, the gRNA is specific for Slc16a11 (SLC16A11) gene or transcript. For example, the gRNA is specific for a sequence with at 70%, at least 80%, at least 90%, at least 95%, at least 98%, at least 99%, or 100% sequence identity to a unique contiguous portion of SEQ ID NO: 2. In some examples, the gRNA is specific to a sequence with at least 90% sequence identity to a unique contiguous portion of SEQ ID NO: 2. In some examples, the gRNA comprises a targeting sequence (sometimes referred to as a spacer) specific to Slc16a11 gene or transcript, for example, by having a targeting sequence that is at least 70%, at least 80%, at least 90%, at least 95%, at least 98%, at least 99%, or 100% complementary to a unique contiguous portion of SEQ ID NO: 2. The targeting sequence of the gRNA is typically about 20 nucleotides, for example, about 15, about 16, about 17, about 18, about 19, about 20, about 21, about 22, about 23, about 24, or about 25 nucleotides. In a specific non-limiting example, the targeting sequence is about 20 nucleotides. The degree of complementarity between a targeting sequence and its corresponding target sequence, when optimally aligned using a suitable alignment algorithm, is about or more than about 50%, about 60%, about 75%, about 80%, about 85%, about 90%, about 95%, about 97.5%, about 98%, about 99%, or more. In some embodiments, the degree of complementarity is about 100%. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some examples, the targeting sequence is at least 80%, at least 85%, at least 90%, at least 91%, at least 92%, at least 93%, at least 94%, at least 95%, at least 96%, at least 97%, at least 98% or at least 99% complementary to a unique, contiguous amino acid sequence about 20 nucleotides long in a Slc16a11 gene or transcript.

In some examples, the gRNA specific for Slc16a11 gene or transcript includes a contiguous sequence at least 70%, at least 80%, at least 90%, at least 95%, at least 98%, at least 99%, or 100% sequence identity to SEQ ID NO: 3. In some examples, the gRNA specific for Slc16a11 gene or transcript includes SEQ ID NO: 3. In some examples, the targeting sequence portion of the gRNA has at least 70%, at least 80%, at least 90%, at least 95%, at least 98%, at least 99%, or 100% sequence identity to SEQ ID NO: 3. In some examples, the targeting sequence portion of the gRNA includes or consists of SEQ ID NO: 3.

In some examples the gRNA is a sgRNA specific for Slc16a11 gene or transcript. In some examples, the sgRNA includes a contiguous sequence having at least 70%, at least 80%, at least 90%, at least 95%, at least 98%, at least 99%, or 100% sequence identity to SEQ ID NO: 3. In some examples, the sgRNA includes SEQ ID NO: 3. In some examples, the targeting sequence portion of the sgRNA has at least 70%, at least 80%, at least 90%, at least 95%, at least 98%, at least 99%, or 100% sequence identity to SEQ ID NO: 3. In some examples, the targeting sequence portion of the sgRNA includes or consists of SEQ ID NO: 3.

Methods of designing gRNA and determining appropriate targeting sequences are have been described (see e.g., Hanna and Doench (2020) Design and analysis of CRISPR-Cas experiments, Nature Biotechnology, 38:813-823(2020) herein incorporated by reference in its entirety). Numerous software tools can be used to design and analyze of CRISPR-Cas experiments, including resources to design optimal g RNAs for various modes of manipulation and to analyze the results of such experiments. Online databases of validated gRNAs are also readily available (see addgene.org/crispr/reference/grna-sequence/and genscript.com/gRNA-database.html).

### II. Nucleic Acids and Expression Vectors

Nucleic acids (e.g., heterologous nucleic acids or isolated nucleic acid molecules, such as DNA, cDNA, RNA (e.g., mRNA)) encoding the RNAi, gRNAs, and/or Cas protein are also provided herein. Nucleic acids can readily be produced using the disclosed sequences provided herein, sequences available in the art, and the genetic code.

Degenerate variants of the disclosed nucleic acid sequences are also disclosed. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA.

Codon preferences and codon usage tables for a particular species can be used to engineer isolated nucleic acid molecules encoding protein products, such as Cas9, that take advantage of the codon usage preferences of that particular species. For example, the nucleic acid can be designed to have codons that are preferentially used by a particular organism of interest (e.g., the organism of origin for a PBMC to be modified, or an organism to be administered the nucleic acid). In some examples, the nucleic acids are codon optimized for expression in human. Thus, in some examples a Cas nuclease (or dead nuclease) sequence is codon optimized for expression in a human PBMC (e.g., T cell, exhausted T cell).

The disclosed nucleic acids can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Nucleic acid sequences can be prepared using any suitable method, including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences may be obtained by the ligation of shorter sequences.

The disclosed nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements). The nucleic acids can also be prepared by amplification methods. Amplification methods include the polymerase chain reaction (PCR), the ligase chain reaction (LCR), the transcription-based amplification system (TAS), the self-sustained sequence replication system (3SR), and the Q0 replicase amplification system (QB). A wide variety of cloning and in vitro amplification methodologies are known to persons skilled in the art.

In some embodiments, the disclosed nucleic acids are included in an expression vector (e.g., viral vector, plasmid, or other vehicle) for expression in a host, or specifically in a target cell (e.g., PBMC, T cell, or exhausted T cell (including a terminally exhausted T cell)). In some examples, the expression vector includes a promoter operably linked to a disclosed nucleic acid molecule. For example, a promoter can be operably linked to an RNAi, gRNA, or Cas nuclease (or dead nuclease) to drive its expression. In some examples, a vector encodes both a Cas nuclease (or dead nuclease) and a gRNA. Additional expression control sequences, such as one or more enhancers, transcription and/or translation terminators, and initiation sequences can also be included in the expression vector. In some embodiments, the disclosed nucleic acids are included in a viral vector. Exemplary viral vectors that can be used include, but are not limited to, polyoma, SV40, adenovirus, vaccinia virus, adeno-associated virus (AAV), herpes viruses including HSV and EBV, Sindbis viruses, alphaviruses and retroviruses of avian, murine, and human origin. Baculovirus (Autographa californica multinuclear polyhedrosis virus; AcMNPV) vectors can also be used. Other suitable vectors include orthopox vectors, avipox vectors, fowlpox vectors, capripox vectors, suipox vectors, lentiviral vectors, alpha virus vectors, and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio. Biologically functional viral and plasmid DNA vectors capable of expression and replication in a cell (e.g., PBMC, T cell, or exhausted T cell (including a terminally exhausted T cell)) are known and a suitable vector can be identified by one of ordinary skill in the art. In some examples, the vector includes a selectable marker (such as an antibiotic resistance gene (e.g., puromycin) or a reporter gene (e.g., green fluorescent protein (GFP)). In other examples, a selectable marker and/or reporter is not included in the vector.

The disclosed nucleic acids can be introduced into a host cell by DNA transfer (e.g., oligonucleotides), or introduced and expressed in a suitable host cell (e.g., expression cassette or vector). In some examples, the expressed product is an RNA (e.g., siRNA or gRNA), in other examples, the expressed product is a protein (e.g., Cas9). The cell may be prokaryotic or eukaryotic. In some embodiments, the host cell is a PBMC (e.g., T cell or exhausted T cell). Methods of transient or stable transfer can be used. Transient transfer indicates that the foreign nucleic acid is only present transiently (e.g., degraded after a period of time, cleared by the host cell, or otherwise not stably replicated). Stable transfer indicates that the foreign nucleic acids is continuously maintained in the host.

To obtain optimal expression of the disclosed nucleic acids, expression cassettes can contain, for example, a strong promoter to direct transcription, a ribosome binding site for translational initiation (e.g., internal ribosomal binding sequences), and a transcription/translation terminator can be used. For expression in E. coli, a promoter, such as the T7, trp, lac, or lamda promoters, a ribosome binding site, and preferably a transcription termination signal can be used. For eukaryotic cells, such as a PBMC, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). Additional operational elements include, but are not limited to, leader sequence, termination codons, polyadenylation signals and any other sequences necessary for the appropriate transcription and subsequent translation of the nucleic acid sequence.

The disclosed nucleic acids or vectors can be introduced into the host cell by any suitable method (e.g., transformation). Numerous methods of transformation are known, such as: chemical methods (e.g., calcium-phosphate transfection), physical methods (e.g., electroporation, microinjection, particle bombardment), fusion (e.g., liposomes), lipofection, nucleofection, receptor-mediated endocytosis (e.g., DNA-protein complexes, viral envelope/capsid-DNA complexes), particle gun accelerator (gene gun), and by biological infection by viruses such as recombinant viruses (Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA. Successfully transformed cells can be selected by resistance to antibiotics conferred by genes contained in the vector, such as the amp, gpt, neo and hyg genes. In some examples, a disclosed nucleic acid (e.g., gRNA) is incorporated in a ribonucleoprotein (RNP) complex (e.g., a gRNA-Cas complex). RNPs can be introduced into a host cell by transformation, for example, by nucleofection.

Modifications can be made to the disclosed nucleic acids without diminishing biological activity of the encoded product. For example, modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

### III. Modified PBMCs

Provided herein are modified peripheral blood mononuclear cells (PBMCs) with reduced expression of Slc16a11, reduced activity of MCT11, or both. In some examples, expression of Slc16a11 is reduced by at least 10%, at least 20%, at least 30%, at least 40%, at least 50%, at least 60%, at least 70%, at least 80%, at least 90%, at least 95%, at least 98%, or at least 100% relative to a suitable control (e.g., a PBMC prior to modification). In some examples, activity of MCT11 is reduced by at least 10%, at least 20%, at least 30%, at least 40%, at least 50%, at least 60%, at least 70%, at least 80%, at least 90%, at least 95%, at least 98%, or 100% relative to a suitable control (e.g., a PBMC prior to modification). Reducing activity includes reducing any measurable biological function of MCT11, for example, by reducing transport of monocarboxylates (e.g., lactic acid) into the modified PBMC. In some examples, the modified PBMC with reduced expression of Slc16a11, reduced activity of MCT11, or both, has increased effector activity (e.g., anti-tumor) relative to a suitable control (e.g., unmodified PBMC). In some examples, the modified PBMC is a T cell, and the T cell has increased resistance to T cell exhaustion relative to a suitable control (e.g., unmodified PBMC).

The modified PBMC can further include additional modifications, for example, the PBMC can express or otherwise contain a chimeric antigen receptor (CAR) or engineered T cell receptor (TCR).

In some examples, the modified PBMC is a T cell, for example, a CD8+ or a CD3+ T cell. The T cell can be reactive to a tumor-specific antigen, for example, CD19, CD20, BCMA, MUC1, PSA, CEA, HER1, HER2, TRP-2, EpCAM, GPC3, mesothelin 1(MSLN), or EGFR. In some examples, the T cell is a tumor-infiltrating lymphocyte (TIL). In some examples, the T cell is a therapeutic T cell, or will be used as a therapeutic T cell, for example, as an ACT therapy. In some examples, the T cell is an exhausted T cell (including terminally exhausted T cells). Exhausted T cells are dysfunctional T cells characterized by a progressive loss of effector function (for example, loss of IL-2, TNF-α, and IFN-γ production) and sustained expression of inhibitory receptors such as PD-1, T cell immunoglobulin domain and mucin domain-containing protein 3 (Tim-3), CTLA-4, lymphocyte-activation gene 3 (LAG-3), and CD160. In some examples, the exhausted T cell is a terminally exhausted T cell, which have high and persistent expression of programmed cell death 1 (PD1^{hi}) and are positive for T cell immunoglobulin and mucin domain-containing protein 3 (TIM3⁺).

In some embodiments, the modified PBMC includes an agent that reduces Slc16a11 expression, for example, one or more of the disclosed inhibitory RNA (RNAi) specific for Slc16a11 gene or transcript, or one or more guide RNA (gRNAs) specific for Slc16a11 gene or transcript (for example in combination with a Cas nuclease or dead Cas nuclease, such as an RNP).

In some examples, the agent that reduces Slc16a11 expression is one or more of the disclosed inhibitory RNA (RNAi), for example, a short hairpin RNA (shRNA), short interfering RNA (siRNA), microRNA (miRNA), or an antisense RNA specific to Slc16a11. In specific, non-limiting examples, the RNAi is a shRNA specific for Slc16a11 gene or transcript, for example, the siRNA is specific to a sequence comprising at least 70%, at least 80%, at least 90%, at least 95%, at least 98%, at least 99%, or 100% sequence identity to SEQ ID NO: 2. In some examples, the shRNA is specific to a sequence with at least 90% sequence identity to a unique, contiguous portion of SEQ ID NO: 2. In some examples, the shRNA has at least 70%, at least 80%, at least 90%, at least 95%, at least 98%, at least 99%, or 100% sequence identity to SEQ ID NO: 4. For example, the shRNA can have at least 90% sequence identity to SEQ ID NO: 4. In some examples, the shRNA consists of or comprises SEQ ID NO: 4.

In some examples, the agent is a disclosed gRNA specific for Slc16a11 gene or transcript, for example, the gRNA is specific for a sequence with at 70%, at least 80%, at least 90%, at least 95%, at least 98%, at least 99%, or 100% sequence identity to SEQ ID NO: 2. For example, the gRNA can be specific to a sequence with at least 90% sequence identity to SEQ ID NO: 2. In some examples, the gRNA comprises a targeting sequence specific to Slc16a11 gene or transcript, for example, by having a targeting sequence that is at least 70%, at least 80%, at least 90%, at least 95%, at least 98%, at least 99%, or 100% complementary to a unique, contiguous portion of SEQ ID NO: 2.

In some examples, the gRNA specific for Slc16a11 gene or transcript includes a contiguous sequence at least 70%, at least 80%, at least 90%, at least 95%, at least 98%, at least 99%, or 100% sequence identity to SEQ ID NO: 3. In some examples, the gRNA specific for Slc16a11 gene or transcript includes SEQ ID NO: 3. In some examples, the targeting sequence portion of the gRNA has at least 70%, at least 80%, at least 90%, at least 95%, at least 98%, at least 99%, or 100% sequence identity to SEQ ID NO: 3. In some examples, the targeting sequence includes or consists of SEQ ID NO: 3. In some examples, the modified PBMC includes a RNP complex that includes the disclosed gRNA and a Cas nuclease, such as Cas9, dCas9, dCas13d, or Cas13d.

In some examples, the modified PBMC includes a heterologous nucleic acid molecule encoding one or more of the disclosed nucleic acids encoding the RNAi (e.g., shRNA, siRNA, antisense RNA) or gRNA. RNAi or gRNA may be encoded as DNA (for example, encoded on a DNA vector), but expressed as RNA. In some examples, the heterologous nucleic acid molecule encodes the disclosed gRNA specific for Slc16a11 and a Cas nuclease (or a dead Cas nuclease). In specific examples, the Cas nuclease is a Cas9 nuclease. In other examples, the Cas nuclease is a Cas13d nuclease.

In some embodiments, the modified PBMC includes the disclosed vector encoding the RNAi or gRNA. Thus, in some examples, the modified PBMC expresses the RNAi or gRNA. If gRNA is used, a Cas nuclease (e.g., Cas9, dCas9, dCas13d, or Cas13d) can also be encoded on the same or different vector, for example, to co-express a Cas nuclease (or dead Cas nuclease) and one or more gRNA specific for Slc16a11 in the modified PBMC. In some examples, the gRNA includes a spacer sequence and DR sequence (such as DR-spacer-DR-spacer) and the Cas nuclease is Cas13d, and Slc16a11 RNA is edited. In some examples, the gRNA includes a crRNA and tracrRNA (expressed either as two separate molecules, or as one fusion molecule, such as a sgRNA) and the Cas nuclease is Cas9. In some examples, the vector includes a cassette including two or more gRNA specific for Slc16a11, wherein the two or more gRNAs have the same or different targeting sequences (e.g., may target two different regions of Slc16a11).

Nucleic acids or vectors can be transiently or stably introduced into a PBMC (e.g., T cell). In a specific, non-limiting example, the vector is stably introduced into the modified PBMC, thereby resulting in stable expression of the RNAi or gRNA in the modified PBMC. In some examples, the nucleic acid encoding the RNAi or gRNA is operably linked to a cell specific promoter (e.g., a T cell specific promoter) in the vector. Expression of the RNAi or gRNA can be constitutive or inducible. Exemplary promoters include NFAT, EF1a, PGK, U6, or H1. In one example, gRNA is expressed from a U6 or H1 promoter. In one example, a Cas nuclease (or dead Cas nuclease) is expressed from a CMV promoter.

In some embodiments, the modified PBMC includes a non-naturally occurring genetic modification that reduces an amount of functional MCT11. Reducing functional MCT11 includes genetic modifications that decrease Slc16a11 expression (e.g., decreasing transcription or translation of Slc16a11 gene or transcript) in the modified PBMC. In some examples, the genetic modification is a non-naturally occurring genetic modification of a Slc16a11 gene. In other examples, the genetic modification is a non-naturally occurring genetic modification of a regulatory element of Slc16a11 (e.g., a promoter, response element, enhancer, transcription factor, or other regulator that affects expression of Slc16a11). The regulatory element can be cis-acting or trans-acting.

In some examples, the non-naturally occurring genetic modification is a modification that reduces an amount of functional MCT11 in the modified PBMC. For example, the genetic modification can result in the production of dysfunctional MCT11. In some examples, the genetic modification results in the production of unstable MCT11, such that the accumulation of functional MCT11 is reduced. The genetic modification can be any non-naturally occurring modification that results in a decreased amount of MCT11. Non-limiting examples of genetic modifications include a point mutation, partial deletion, full deletion, or insertion.

In some embodiments, the modified PMBC includes an inhibitor of MCT, for example, a small molecule inhibitor. In some examples, the small molecule inhibitor of MCT is one or more of 7ACC1, AR-C155858, UK 5099, SR 13800, CHC, AR-C 141990 hydrochloride, AZD3965, or BAY8002. In a specific, non-limiting example, the modified PBMC includes a small molecule inhibitor of MCT11.

### Methods of Generating Modified PBMCs

Also provided herein are methods of generating the disclosed modified PBMCs by introducing the agent, non-naturally occurring genetic modification, or inhibitor into a PBMC, thereby generating the modified PBMC with reduced expression of Slc16a11 and/or reduced activity of MCT11. In some embodiments, the PBMC is obtained from a subject before introducing the agent, non-naturally occurring genetic modification, or inhibitor. PBMCs can be harvested or isolated, for example, from a blood sample, such as a venous blood sample, from the subject. Several techniques for isolating PBMCs are known, for example, density centrifugation (the Ficoll approach), isolation by cell preparation tubes (CPTs), or isolation by SepMate™ tubes. In some examples, aphersis or leukapheresis is used to harvest PBMCs. Erythrocyte contamination can be evaluated, for example, by microscopic analysis of the sample. Flow cytometry techniques (e.g., FACS) can be used to assess the composition of the isolated PBMC populations, for example, to identify monocytes (e.g., CD14), T cells (e.g., CD3, CD8, CD4), B cells (e.g., CD20), or NK cells (e.g., CD56). FACS techniques can also be used to enrich or deplete a particular cell type from a PBMC (for example, enrich or deplete CD14, CD3, CD8, CD4, CD28, CD20, CD56, or combinations thereof).

In some examples, T cells are isolated from a PBMC sample, or the PBMC sample is enriched for T cells, for example, isolated or enriched for CD3⁺ or CD8⁺ T cells. In some examples, a sample is enriched by negative selection, for example, by selecting and removing unwanted cell types from a sample (e.g., cell types other than T cells, and/or naïve or memory T cells). In some examples, FACS is used to enrich for a particular PBMC, for example, to enrich for T cells (e.g., CD3 or CD8 positive T cells). FACS can also be used to assess whether exhausted T cells, or specifically terminally exhausted T cells (PD-1^{hi}, TIM3⁺), are present in a PBMC sample, or sort a PBMC sample to enrich for exhausted T cells (including terminally exhausted T cells), or conversely remove exhausted T cells. Antigen responsiveness of the PBMCs can be assessed, for example, by measuring release of cytokines, e.g., IFNγ, IL-10, IL-6, IL-8 and TNFα.

In some examples, the PBMCs are obtained from a subject to be treated, such as a subject having cancer. In other examples, the PBMCs are obtained from a donor subject, such as a subject who does not have cancer. In some examples, exhausted T cells are obtained from a tumor biopsy or sample (e.g., tumor infiltrating lymphocytes).

In some examples, the agent, non-naturally occurring genetic modification, or inhibitor is introduced into a PBMC ex vivo. In such examples, such methods can further include selecting modified PBMCs having reduced expression of Slc16a11, reduced activity of MCT11, or both (such as purifying or isolating such cells away from cells not having reduced expression of Slc16a11, not having reduced activity of MCT11, or both). Such methods can also further include selecting modified PBMCs that are T cells, for example, T cells that are CD3+ or CD8+. Exemplary selection methods include using flow cytometry, panning or magnetic separation. The disclosed methods in some examples further include introducing the selected modified PBMCs having reduced expression of Slc16a11, reduced activity of MCT11, or both, into a subject, such as a subject with a cancer to be treated with the selected modified PBMCs having reduced expression of Slc16a11, reduced activity of MCT11, or both.

In some examples, the agent, non-naturally occurring genetic modification, or inhibitor is administered to the subject, and the agent, non-naturally occurring genetic modification, or inhibitor is introduced into a PBMC (e.g., T cells or exhausted T cells (including terminally exhausted T cells)) in vivo.

In some embodiments, the method of generating the modified PBMC further includes selecting a PBMC or cell type (e.g., T cells, exhausted T cells (including terminally exhausted T cells)), for example, from a sample (e.g., tumor biopsy, blood, population of T cells) before introducing the inhibitor, agent, or non-naturally occurring genetic modification. In some examples, the selected PBMC is reactive to a tumor-specific antigen, for examples, one or more of: CD19, CD20, BCMA, MUC1, PSA, CEA, HER1, HER2, TRP-2, EpCAM, GPC3, mesothelin 1(MSLN), or EGFR. In some examples the selected PBMC is a T cell. In some examples, the T cell is CD8+ or CD3+. In some examples, the T cell is an adoptive cell transfer (ACT) therapy T cell, for example, the selected exhausted T cell can include a chimeric antigen receptor (CAR) or an engineered T cell receptor (TCR) specific for a tumor antigen. In further examples, the selected PBMC is a tumor-infiltrating lymphocyte (TIL). In some examples, the T cell is an exhausted T cell, such as a terminally exhausted T cell, which highly expresses programmed cell death 1 (PD1^{hi}) and is positive for T cell immunoglobulin and mucin domain-containing protein 3 (TIM3⁺).

In some examples, the agent (RNAi or gRNA) or inhibitor (e.g., small molecule inhibitor) is introduced, for example, by contacting a PBMC with the agent or inhibitor, thereby generating the modified PBMC. In other examples, the inhibitor or agent is introduced by transfecting or transforming a PBMC with the disclosed nucleic acid molecule encoding the inhibitor or agent or the vector encoding a disclosed nucleic acid molecule, thereby generating the modified PBMC. Methods of transforming or transfecting a host cell are described herein, and can include: chemical methods (e.g., calcium-phosphate transfection), physical methods (e.g., electroporation, microinjection, particle bombardment), fusion (e.g., liposomes), nucleofection, receptor-mediated endocytosis (e.g., DNA-protein complexes, viral envelope/capsid-DNA complexes) and by biological infection by viruses, such as recombinant viruses. In the case of infection by retroviruses, the infecting retrovirus particles are absorbed by the target cells, resulting in reverse transcription of the retroviral RNA genome and integration of the resulting provirus into the cellular DNA. In some examples, a ribonucleoprotein (RNP) complex including the gRNA and a Cas nuclease or dead nuclease (e.g., Cas9 or Cas13d) is directly introduced into the PBMC. Methods of introducing a RNP complex into a host cell have been described. For example, the PBMC can be nucleofected with the RNP. In some examples, the PBMC is transfected with the RNP by electroporation (see e.g., Seki and Rutz, (2018) J Exp Med. 215(3): 985-997). In some examples, lipid-containing oligoaminoamides (lipo-OAAs) are used to as a carrier for intracellular delivery of the RNP complex (see e.g., Kuhn et al. (2020) Bioconjugate Chem. 31(3):729-742).

In specific, non-limiting examples, the introduced agent is shRNA, and the shRNA is introduced into the PBMC through infection with a viral vector encoding the shRNA. Introduction by a viral vector allows for stable integration of shRNA and long-term knockdown of the targeted gene. In another specific, non-limiting example, the introduced agent is siRNA, and siRNA is introduced cytosolically into a host cell capable of transfection.

In some embodiments, the non-naturally occurring genetic modification is introduced into the PBMC. The genetic modification can be any non-naturally occurring modification that results in decreased expression of Slc16a11 or reduced activity of MCT11. Non-limiting examples of genetic modifications include a point mutation, partial deletion, full deletion, or insertion. In some examples, the genetic modification is induced by a targeted genome editing technique, such as CRISPR/Cas, zinc finger nuclease, or TALEN modification of a Slc16a11 gene. Methods of genome editing have been previously described, for example, in Nemudryi et al. (2014) Acta Naturae; 6(3): 19-40, herein incorporated by reference in its entirety. In some examples, the genetic modification reduces Slc16a11 expression, for example, by at least 20%, at least 30%, at least 40%, at least 50%, at least 60%, at least 70%, at least 80%, at least 90%, at least 95%, at least 98%, or 100%. In some examples, the genetic modification reduces MCT11 activity, for example, by at least 20%, at least 30%, at least 40%, at least 50%, at least 60%, at least 70%, at least 80%, at least 90%, at least 95%, at least 98%, or 100%. In some examples, the genetic modification reduces lactic acid transport activity of MCT11, for example, by at least 20%, at least 30%, at least 40%, at least 50%, at least 60%, at least 70%, at least 80%, at least 90%, at least 95%, at least 98%, or 100%.

In some embodiments, the modified PBMC is incubated with at least one cytokine selected from the group consisting of interleukin 2 (IL-2), interleukin 7 (IL-7), and interleukin 15 (IL-15).

In some embodiments, introducing the agent, non-naturally occurring genetic modification, or inhibitor reduces activity of MCT11 in the modified PBMC by at least 10%, at least 20%, at least 30%, at least 40%, at least 50%, at least 60%, at least 70%, at least 80%, at least 90%, or at least 100% relative to a suitable control (e.g., an unmodified PBMC). In some examples, the genetic modification reduces lactic acid transport activity of MCT11, for example, by at least 20%, at least 30%, at least 40%, at least 50%, at least 60%, at least 70%, at least 80%, at least 90%, at least 95%, at least 98%, or 100% relative to a suitable control. In some examples, introducing the agent, non-naturally occurring genetic modification, or inhibitor reduces protein levels of MCT11 by at least 10%, at least 20%, at least 30%, at least 40%, at least 50%, at least 60%, at least 70%, at least 80%, at least 90%, at least 100% or more relative to a suitable control. In some examples, introducing the agent, non-naturally occurring genetic modification, or inhibitor reduces activity of MCT11 by at least 10%, at least 20%, at least 30%, at least 40%, at least 50%, at least 60%, at least 70%, at least 80%, at least 90%, at least 100% or more relative to a suitable control. Reducing activity includes reducing any measurable biological function of MCT11, for example, by reducing transport of monocarboxylates (e.g., lactic acid) into the modified PBMC.

In some embodiments, decreasing expression of Slc16a11 or activity of MCT11 in a PBMC increases effector function, reduces exhaustion, increases resistance to exhaustion, or combinations thereof. In some examples, the PBMC is a T cell, and decreasing expression of Slc16a11 or activity of MCT11 in the PBMC increases effector function of the T cell, reduces exhaustion of the T cell, or both. In further examples, the PBMC is a T cell and decreasing expression of Slc16a11 or activity of MCT11 in the PBMC increases resistance to T cell exhaustion. In some examples, the disclosed modified PBMCs, such as modified T cells, do not become exhausted (e.g., do not become PD1^{hi} and TIM3⁺). In some examples, the disclosed modified PBMCs, such as modified T cells, become exhausted at a slower rate, for example the number of days to progress to an exhausted cell (e.g., PD1^{hi} and TIM3⁺) is increased by at least 10%, at least 25%, at least 50%, at least 75%, at least 90%, at least 95%, or at least 99%, for example relative to a PBMC/T cell with native MCT11 expression/activity. In some examples, the disclosed modified PBMCs, such as modified T cells, results in a population of modified PBMCs, such as modified T cells, with fewer exhausted cells (e.g., PD1^{hi} and TIM3⁺), such as a reduction of at least 10%, at least 25%, at least 50%, at least 75%, at least 90%, at least 95%, or at least 99%, for example relative to a PBMC/T cell with native MCT11 expression/activity.

### IV. Pharmaceutical Compositions

Also disclosed herein are pharmaceutical compositions useful for treating cancer or increasing response to immunotherapy. In some examples, the pharmaceutical composition includes (1) one or more of: the disclosed RNAi specific to Slc16a11, one or more gRNAs specific for Slc16a11, the nucleic acid or vector encoding the RNAi or gRNA, the inhibitor (e.g., MCT11 inhibitor), or the modified PBMC; and (2) a pharmaceutically acceptable carrier. In specific, non-limiting examples, the pharmaceutical composition includes a modified PBMC and a pharmaceutically acceptable carrier.

In some examples, the pharmaceutical composition includes (1) one or more of: the RNAi specific to Slc16a11, the gRNAs specific for Slc16a11, the nucleic acid or vector encoding the RNAi or gRNA, the MCT11 inhibitor, or the modified PBMC; (2) a cancer immunotherapy; and (3) a pharmaceutically acceptable carrier. In some examples, the cancer immunotherapy is an ACT therapy (e.g., CAR-T, TCR, TIL), a monoclonal antibody (e.g., anti-PD-1, anti-EGFR, anti-CTLA4), a T cell agonist antibody, or an oncolytic virus. In a specific, non-limiting example, the pharmaceutical composition includes the modified PBMC, an antibody cancer immunotherapy, and a pharmaceutically acceptable carrier. In another, non-limiting example, the pharmaceutical composition includes: one or more of the RNAi specific to Slc16a11, the gRNAs specific for Slc16a11, the nucleic acid or vector encoding the RNAi or gRNA; an ACT immunotherapy (e.g., CAR-T, TCR, TIL); and a pharmaceutically acceptable carrier.

A “pharmaceutically acceptable carrier” includes any and all solvents, dispersion media, coatings, antibacterial and antifungal agents, isotonic and absorption delaying agents, and the like, compatible with pharmaceutical administration (see, e.g., Remington's Pharmaceutical Sciences, 23rd Edition, Academic Press, Elsevier, (2020)). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions include those provided in Remington's Pharmaceutical Sciences, 23rd Edition, Academic Press, Elsevier, (2020). In some examples, the pharmaceutical composition is formulated for intravenous administration.

### V. Methods of Treating Cancer

Also disclosed herein are methods of treating cancer or a tumor in a subject by administering an effective amount of a disclosed composition (the RNAi specific to Slc16a11, the gRNA specific to Slc16a11 and a Cas nuclease or dead Cas nuclease (which may be administered as an RNP complex), a nucleic acid or vector encoding the RNAi or gRNA (wherein in some examples the vector also expresses and a Cas nuclease or Cas dead nuclease), the MCT11 inhibitor, the modified PBMC, or the pharmaceutical composition disclosed herein (hereinafter collectively referred to as “composition”)), to the subject, thereby treating the cancer or tumor. In a specific, non-limiting example, the administered composition is an effective amount of the modified PBMCs disclosed herein. In some examples, PBMCs are removed from the subject and modified as disclosed herein ex vivo, then the modified cells are introduced into the subject. In some examples, PBMCs are modified in vivo, for example by introducing a therapeutic molecule provided herein (e.g., RNAi specific to Slc16a11, gRNA specific for Slc16a11) into the subject.

Also disclosed herein are methods of increasing a response to immunotherapy in a subject by administering an effective amount of the disclosed composition, thereby increasing a response to immunotherapy. In a specific, non-limiting example, the method is a method of increasing a response to immunotherapy in a subject and the composition is the disclosed vector encoding the RNAi or gRNA, or an MCT11 inhibitor.

In some examples, the subject has a tumor or cancer. In some examples, the subject has a solid tumor or cancer, such as breast carcinomas (e.g. lobular and duct carcinomas, such as a triple negative breast cancer), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma (such as an HPV-positive HNSCC), and lymphatic tumors (including B-cell and T-cell malignant lymphoma).

In some examples, the subject has a liquid tumor or cancer, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

In a non-limiting example, the subject has leukemia, colorectal cancer, cervical cancer, lung cancer, bladder cancer, head and neck cancer, pancreatic cancer, glioblastoma, head and neck squamous cell carcinoma, ovarian cancer, uterine cancer, prostate cancer, breast cancer, melanoma, non-small cell lung cancer (NSCLC), renal cell carcinoma, sarcomas, or adrenal carcinoma. In another non-limiting example, the subject has melanoma.

In some embodiments, the subject is receiving, has received, or will receive immunotherapy, for example, a checkpoint inhibitor targeting PD-1, PD-L1, CTLA-4, CDK4, and/or CDK6. Exemplary checkpoint inhibitors include ipilimumab, nivolumab, pembrolizumab, atezolizumab, avelumab, durvalumab, cemiplimab, palbociclib, ribociclib, and abemaciclib. In some examples, the effective amount of the composition is an amount that increases a response of the subject to an immunotherapy (e.g., a checkpoint inhibitor or ACT); for example, an amount that when administered with the immunotherapy, is more effective at treating cancer or a tumor relative to administration of the immunotherapy (or composition) alone. In some examples, the effective amount is an amount that is synergistic when administered with an immunotherapy, for example, an amount that synergistically prevents, treats, reduces, and/or ameliorates one or more sign or symptom of cancer.

In some examples, the effective amount of the composition is an amount sufficient to prevent, treat, reduce, and/or ameliorate one or more signs or symptoms of cancer in the subject. For example, an amount sufficient to reduce tumor size or tumor load in the subject by at least 10%, at least 20%, at least 30%, at least 40%, at least 50%, at least 60%, at least 70%, at least 80%, at least 90%, at least 95%, or 100%, as compared to a baseline measurement for the same subject, or a suitable control. In some examples, the effective amount is an amount sufficient to inhibit or slow metastasis in the subject. For example, by decreasing tumor spread in the subject by at least 10%, at least 20%, at least 30%, at least 40%, at least 50%, at least 60%, at least 70%, at least 80%, at least 90%, at least 95%, or 100% as compared to a baseline measurement for the same subject, or a suitable control. In some examples, the effective amount is an amount that increases life expectancy of the subject, for example, by at least 10%, at least 20%, at least 30%, at least 40%, at least 50%, at least 60%, at least 70%, at least 80%, at least 90%, at least 100%, at least 200%, at least 400%, or more. In other examples, the effective amount is an amount sufficient to reduce tumor density in the subject, for example, by at least 10%, at least 20%, at least 30%, at least 40%, at least 50%, at least 60%, at least 70%, at least 80%, at least 90%, or 100% as compared to a baseline measurement for the same subject or other suitable control. Non-limiting examples of suitable controls include untreated subjects or subjects not receiving the composition (e.g., subjects receiving other agents or alternative therapies). In further examples, the effective amount is an amount sufficient to target and eliminate tumor cells, for example, eliminate at least 10%, at least 20%, at least 30%, at least 40%, at least 50%, at least 60%, at least 70%, at least 80%, at least 85%, at least 90%, at least 95%, at least 98%, or even 100%, relative to a suitable control.

In some examples, the method reduces expression of Slc16a11 or activity of MCT11 in a target tissue or cell in the subject, for example, in a PBMC, T cell, or exhausted T cell (including terminally exhausted T cells)). In some examples, expression of Slc16a11 or activity of MCT11 is decreased by at least 10%, at least 20%, at least 30%, at least 40%, at least 50%, at least 60%, at least 70%, at least 80%, at least 90%, at least 95%, or 100% relative to a suitable control (e.g., an untreated subject or a baseline reading of the same subject prior to treatment). In some examples, the method reduces protein levels of MCT11 (or functional MCT11), for example, by at least 10%, at least 20%, at least 30%, at least 40%, at least 50%, at least 60%, at least 70%, at least 80%, at least 90%, at least 95%, or 100% relative to a suitable control (e.g., an untreated subject or a baseline reading of the same subject prior to treatment). In some examples, the method reduces expression of Slc16a11 or accumulation of mRNA transcripts by at least 10%, at least 20%, at least 30%, at least 40%, at least 50%, at least 60%, at least 70%, at least 80%, at least 90%, at least 95%, or 100% relative to a suitable control (e.g., an untreated subject or a baseline reading of the same subject prior to treatment).

In some examples, decreasing expression of Slc16a11 or activity of MCT11 increases T cell effector function or decreases T cell exhaustion. In some examples, decreasing expression of Slc16a11 or activity of MCT11 reduces (including prevents or inhibits) T cell exhaustion or increases resistance to (including prevents or inhibits) T cell exhaustion. In some examples, increasing T cell response or reducing T cell exhaustion in a subject increases response to an immunotherapy in the subject.

In a specific, non-limiting example, the method includes administering to the subject the modified PBMC and a pharmaceutically acceptable carrier. When the disclosed PBMC is administered, the composition includes about 10⁴ to 10¹² of the modified PBMCs (for example, about 10⁴-10⁸ cells, about 10⁶-10⁸ cells, about 10⁶-10¹² cells, about 10⁸-10¹² cells, or about 10⁹-10¹⁰ cell). For example, the composition may be prepared such that about 10⁴ to 10¹⁰ modified PBMCs (e.g., about 10⁴, 10⁵, 10⁶, 10⁷, 10⁸, 10⁹, or 10¹⁰ cells/kg) are administered to a subject. In some examples, about 10¹⁰ cells/kg are administered to the subject. In specific examples, the composition includes at least 10⁴, 10⁵, 10⁶, 10⁷, 10⁸, 10⁹, or 10¹⁰ modified PBMCs. In a specific, non-limiting example, about 10⁸-10¹⁰ modified PBMCs are administered to the subject. An appropriate dose can be determined by a skilled clinician based on factors such as the subject, the cancer being treated, treatment history, tumor load and type, clinical stage and grade of the disease, overall health of the subject, and other factors. In some examples, the subject is administered a small molecule inhibitor of MCT (e.g., MCT11) before, after, or substantially at the same time as the modified PBMCs.

In some examples, non-modified lymphocytes are depleted in the subject prior to administering the disclosed composition. In some examples, the subject is also administered one or more cytokine(s) (such as IL-2, IL-7, IL-15, IL-21, and/or IL-12), for example, to support survival and/or growth of the disclosed modified PBMCs and/or an additional ACT therapy administered in combination, in the subject. In a specific, non-limiting example, at least one of IL-2, IL-7, and IL-15 is also administered to the subject. The cytokine(s) are administered before, after, or substantially simultaneously with the composition. In specific examples, at least one cytokine (e.g., IL-2, IL-7, and/or IL-15) is administered simultaneously, for example, with the composition. In some examples, the modified PBMC is reactive to a tumor-specific antigen in the subject having cancer. In some examples, the antigen is one or more of: CD19, CD20, BCMA, MUC1, PSA, CEA, HER1, HER2, TRP-2, EpCAM, GPC3, mesothelin 1(MSLN), or EGFR.

Administration of any of the disclosed compositions can be local or systemic. Exemplary routes of administration include, but are not limited to, oral, injection (such as subcutaneous, intramuscular, intradermal, intraperitoneal, intravenous), sublingual, rectal, transdermal (for example, topical), intranasal, vaginal, and inhalation routes. In some examples, the agent is injected or infused into a tumor, or close to a tumor (local administration), or administered to the peritoneal cavity. Appropriate routes of administration can be determined by a skilled clinician based on factors such as the subject, the condition being treated, and other factors.

Multiple doses of the composition can be administered to a subject. For example, the compositions can be administered daily, every other day, twice per week, weekly, every other week, every three weeks, monthly, or less frequently. A skilled clinician can select an administration schedule based on the subject, the condition being treated, the previous treatment history, and other factors.

In some examples, the subject receives a treatment in addition to the composition, such as one or more of surgery, radiation, chemotherapy, biologic therapy, immunotherapy, or other therapeutic. Exemplary chemotherapeutic agents include (but are not limited to) alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocrotical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include adriamycin, melphalan (Alkeran®) Ara-C (cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), trastuzumab (Herceptin®), irinotecan (CPT-11), leustatin, navelbine, rituximab (Rituxan®) imatinib (STI-571), Topotecan (Hycamtin®), capecitabine, ibritumomab (Zevalin®), and calcitriol. A skilled clinician can select appropriate additional therapies (from those listed here or other current therapies) for the subject, depending on factors such as the subject, the cancer being treated, treatment history, and other factors.

In some examples, the subject is administered an additional therapeutic, such as a monoclonal antibody cancer immunotherapy (e.g., anti-CTLA-4, anti-PD1, or anti-PDL1), a T cell agonist antibody, an oncolytic virus, an adoptive cell transfer (ACT) therapy, or any combination of two or more thereof. The administration of an additional therapeutic may be before, after, or substantially simultaneously with the administration of the disclosed composition. In some examples, the additional therapeutic is a cell cycle or checkpoint inhibitor. In some examples, the checkpoint inhibitor targets PD-1, PD-L1, CTLA-4, CDK4, and/or CDK6. Exemplary inhibitors include ipilimumab, nivolumab, pembrolizumab, atezolizumab, avelumab, durvalumab, cemiplimab, palbociclib, ribociclib, and abemaciclib.

In some examples, the subject is administered an ACT therapy, for example, a chimeric antigen receptor (CAR)-expressing T cell, engineered TCR T cell, or a tumor-infiltrating lymphocyte (TIL). In some examples, the subject is administered an effective amount of the composition and the ACT therapy, and an effective amount of the composition is an amount that increases effectiveness of the ACT (e.g., increases elimination of cancerous cells relative to ACT therapy alone).

The additional therapeutic may be administered substantially simultaneously with the disclosed composition. In some examples, the additional therapeutic is administered prior to administering the composition, for example, at least 1 day, at least 2 days, at least 3 days, at least 4 days, at least 5 days, at least 6 days, at least 7 days, at least 8 days, at least 9 days, at least 12 days, at least 14 days, at least three weeks, at least four weeks, at least one month, or more prior. Multiple doses of the additional therapeutic can be administered to a subject, for example, administered twice daily, once daily, every other day, twice per week, weekly, every other week, every three weeks, monthly, or less frequently. A skilled clinician can select an administration schedule based on the subject, the condition being treated, the previous treatment history, tumor load and type, clinical stage and grade of the disease and overall health of the subject, and other factors.

### VI. Kits

Also provided are compositions and kits that can be used with the disclosed methods. In some examples, the composition or kit includes one or more of the RNAi specific to Slc16a11, the gRNA specific to Slc16a11, a nucleic acid or vector encoding the RNAi or gRNA, the MCT11 inhibitor, and the modified PBMC, for example with a pharmaceutically acceptable carrier. In some examples, the kit includes one or more gRNA specific for Slc16a11 and a Cas nuclease or Cas dead nuclease (which may be an RNP complex). In a specific, non-limiting example, the kit includes a vector encoding one or more gRNA specific for Slc16a11, which can further encode a Cas nuclease or Cas dead nuclease. In some examples, the kit includes one or more of the disclosed RNAi specific for Slc16a11. In further examples, the kit includes the disclosed modified PBMCs.

The kit can include additional reagents, such as one or more of anti-CD3, anti-CD28, IL-2, and IL-15. In some examples, the reagents are present in separate containers. In one example, anti-CD3 and anti-CD28 are in the same container, and may be present, for example, on a bead. In some examples, the kit further includes one or more of a transfection reagent, culture medium, antibiotic, cytokines (e.g., IL-2, IL-15, and IL-7), optionally wherein such reagents are present in separate containers. In some examples the kit or composition includes media in which the PBMCs can be cultured or expanded ex vivo, such as AIM V® media.

### Example 1

### Materials and Methods

This example provides the materials and methods used to generate the data discussed in the Examples below.

### RNA-seq

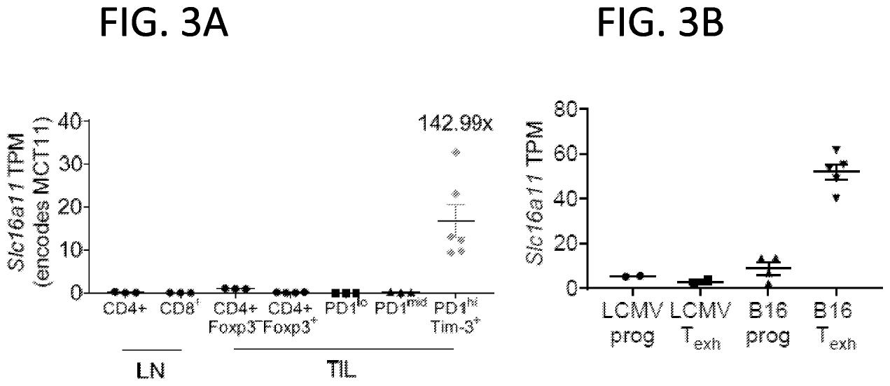

C57/BL6 mice were implanted with B16 melanoma. When tumors reached 7 mm in any direction, lymph node and tumor were harvested and processed, and CD8⁺ T cells were sorted on the basis of CD44, PD-1, and Tim-3 expression from the lymph node (LN) and tumor infiltrating lymphocytes (TIL). RNA-seq was performed on 1,000 cells isolated from the following compartments: LN CD44^{hi}, TIL PD-1^{lo}, TIL PD-1^{mid}, TIL PD-1^{hi}, and TIL PD-1^{hi}Tim3⁺. CD4⁺ T cells from LN and TIL were sequenced from a separate experiment. RNA was prepared from cell lysates of 1000 cells using the Clontech SMARTer® kit, and sequenced on an Illumina NextSEQ®. TPMs were calculated after aligning to the mouse genome (mm9 assembly). Transcripts per million (TPM) plots of Slc16a11 (encoding MCT11) are shown (FIG. 3A).

### Lactic Acid Uptake

C57/BL6 mice were implanted with B16 melanoma as above, and TIL preparations were made. TIL was loaded with pHrodo® Red, a pH sensitive dye, and incubated in Hank's Balanced Salt Solution (HBSS). Lactic acid was pulsed for 30 mins and pH change was measured by flow cytometry, as described in Watson et al. (2021) Nature, 591: 645-651, herein incorporated by reference in its entirety.

### Gain of Function and Loss of Function Mutants

OT-I (OVA-specific) Thy1.1+ congenic T cells were activated with cognate peptide and splenocytes overnight before being retrovirally transduced with shRNA constructs targeting Slc16a11, which were then transferred into congenically mismatched (Thy1.2+) hosts bearing B16-OVA tumors. After 8 days, TIL were harvested and stained for PD-1 and Tim-3 as a readout of terminal differentiation. Separate TIL preparations were also restimulated with OVA peptide overnight in the presence of brefeldin A, then stained intracellularly for IFN-γ and TNFα to measure cytokine production.

### Conditional Knock-Out of MCT11 (Slc16a11)

Conditional knock-out mice for Slc16a11 (MCT11) were generated (MCT11^{COIN}×CD4^{cre}) Wild-type or MCT11 conditional knock-out (MCT11^{COIN}×CD4^{cre}) mice were inoculated with B16 tumor cells. Tumor growth was followed. T cell infiltration (percent live CD8+ cells per tumor area) and cytokine production after restimulation were also recorded.

### Cas9-gRNA Nucleofection

OT-I T cells were isolated from TCR-Tg mice and nucleofected with a Cas9:gRNA ribonucleoprotein complex targeting Slc16a11 (the gRNA of SEQ ID NO: 7) using a Lonza 4D-Nucleofector®. Cells were then activated with anti-CD3/CD28 and expanded to therapeutic quantities in vitro using recombinant IL-2. In parallel, host mice were inoculated with B16-OVA cells. When tumors reached 3 mm in diameter, they were treated with the expanded T cells (10⁷ cells per mouse). Tumor sizes were measured three times weekly until tumors reached 15 mm in any direction, or after 30 days of experimentation.

### Example 2

### Discovery of MCT11 on Exhausted T Cells

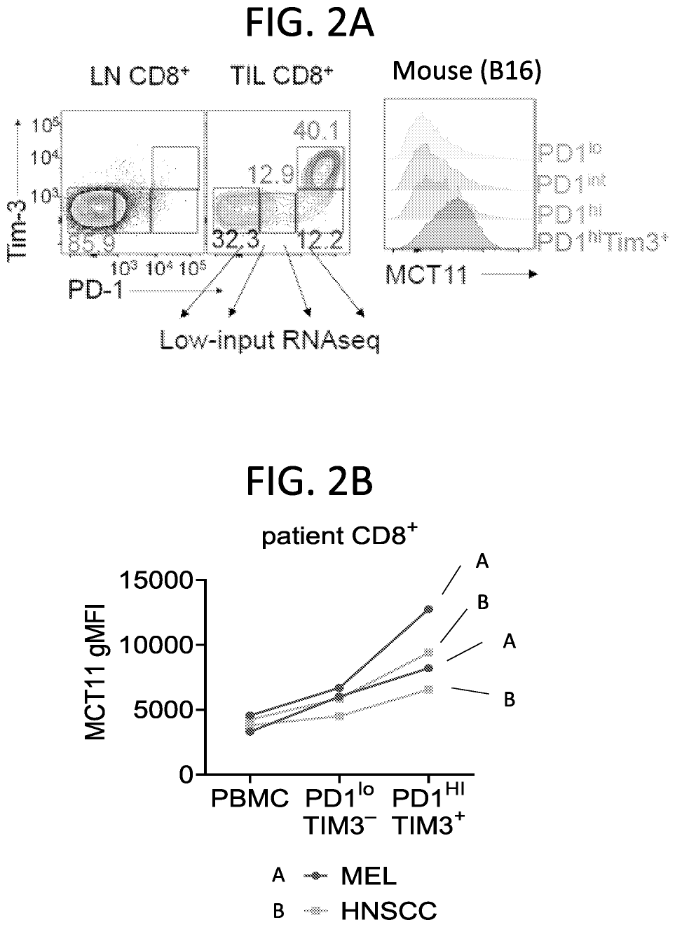

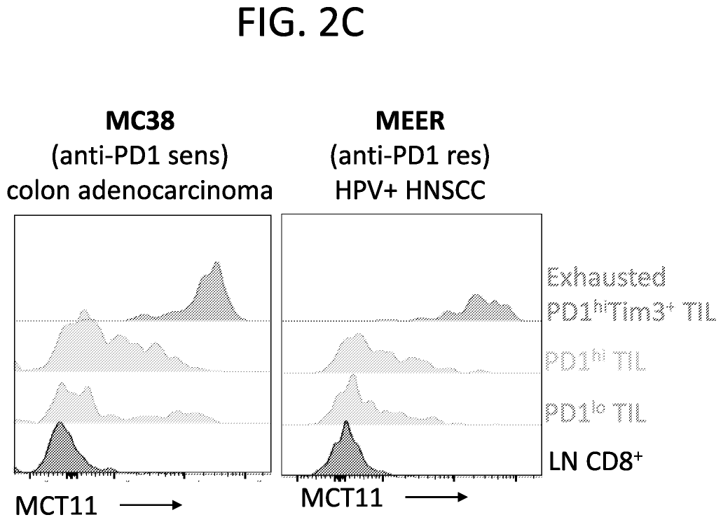

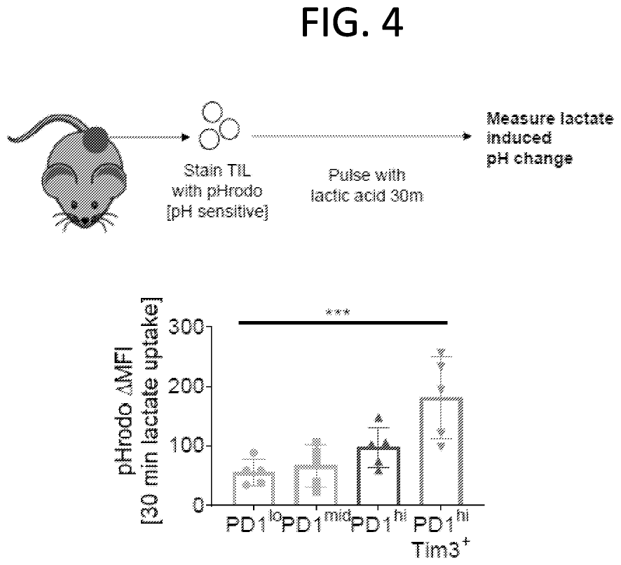

Using RNA-seq and metabolic profiling, terminally exhausted T cells (dysfunctional T cells common in cancer environments) were found to highly express a novel nutrient transporter called MCT11 (encoded by Slc16a11) (FIGS. 3A and 3B). MCT11 likely transports monocarboxylates, short chain carbon sources such as lactic acid, pyruvate, and short-chain fatty acids. MCT11 upregulation in exhausted T cells in human and mice was confirmed by flow cytometry and RNA-Seq (see, FIGS. 2A-2C and FIGS. 3A-3B). Further, it was confirmed that terminally exhausted T cells specifically take up monocarboxylates, such as lactic acid (FIG. 4). However, MCT11 is not expressed on the surface of exhausted T cells induced by chronic viral infection. These findings suggest that MCT11 may be important in providing nutrient flux to terminally exhausted T cells.

### Example 3

### MCT11 Gain of Function and Loss of Function

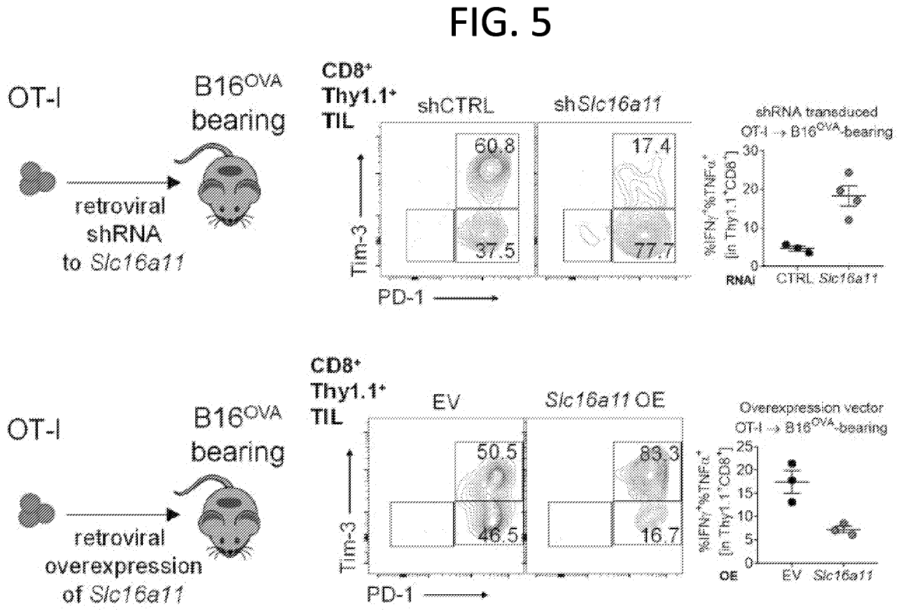

T cells in which MCT11 was knocked down did not proceed fully to exhaustion (PD1^{hi} but not Tim-3⁺) and produced more IFNγ and TNFα in response to peptide stimulation (FIG. 5). In a companion experiment, OT-I T cells were transduced with a retroviral overexpression vector encoding Slc16a11. Those T cells rapidly progressed to exhaustion (PD1^{hi}Tim3⁺) and demonstrated poor cytokine production (FIG. 5). Thus, deletion of MCT11 in tumor-specific T cells transferred into tumor-bearing mice resulted in increased T cell function and decreased exhaustion, while overexpression of MCT11 in tumor-specific T cells accelerated the development of the exhausted, dysfunctional phenotype.

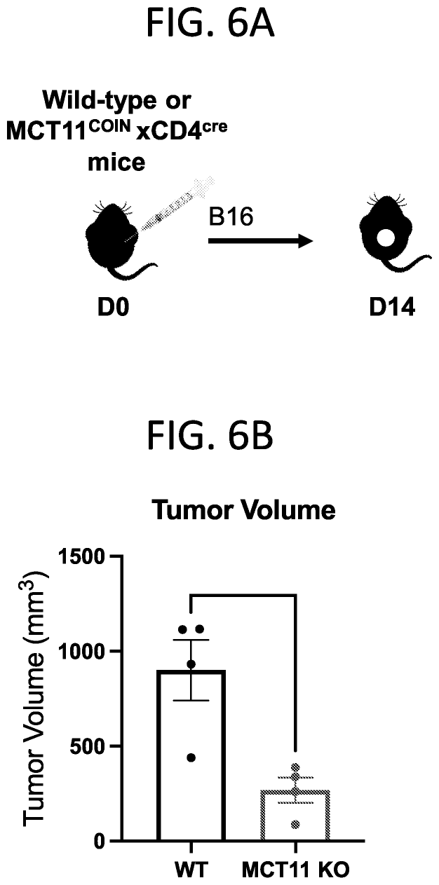

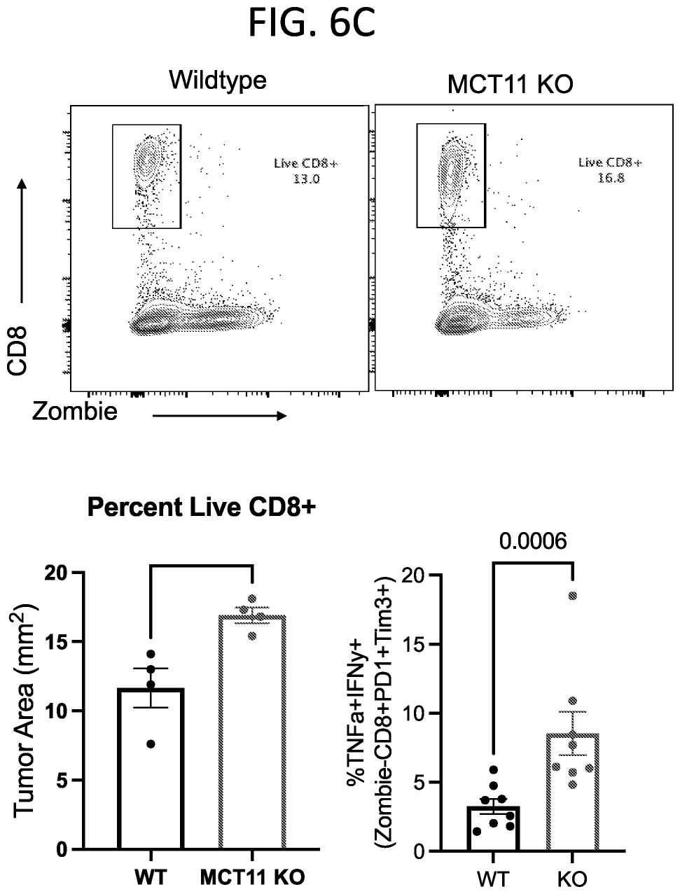

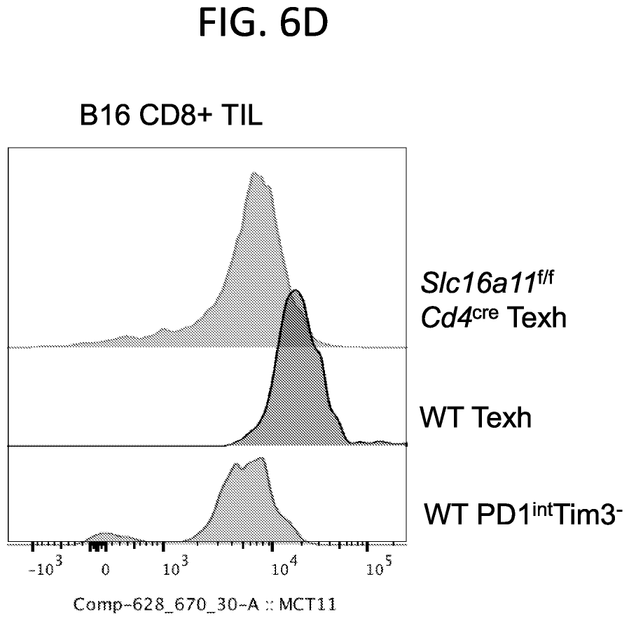

Tumor growth and T cell function were investigated in mice carrying a conditional deletion of MCT11 (Slc16a11) in T cells (MCT11^{COIN}×CD4^{cre}). Wild-type (WT) and MCT11^{COIN}×CD4^{cre} mice were inoculated with B16 melanoma (FIG. 6A). The MCT11^{COIN}×CD4^{cre} mice had smaller tumor volume (FIG. 6B), increased T cell infiltration, and increased T cell function (as measured by cytokine production after restimulation) (FIG. 6C) relative to WT. MCT11 knock-out was confirmed by antibody staining; a MCT11 mAb did not stain exhausted T cells in mice bearing the conditional deletion of MCT11 (FIG. 6D).

### Example 4

### CRISPR/Cas9 Mediated Deletion of Slc16a11

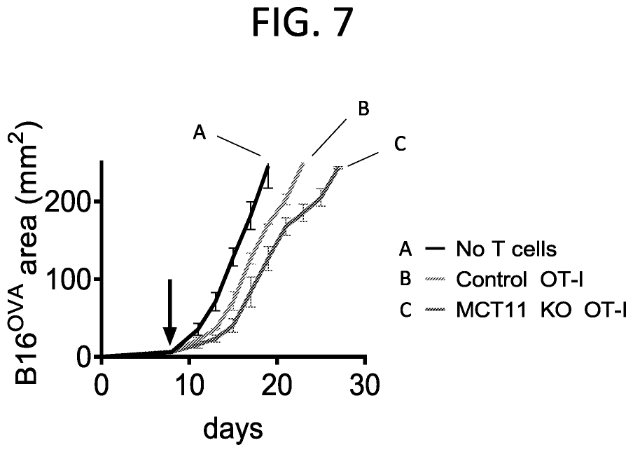

OT-I OVA-specific T cells were nucleofected with Cas9:gRNA ribonucleoprotein complexes to delete Slc16a11. Mice that were transferred the MCT11 knock-out T cells (MCT11 KO OT-I) had smaller tumor area than either control (no T cell control, or treated with control OT-I cells) (FIG. 7). Thus, the MCT11 KO T cells were superior therapeutic cells after just one dose, and showed lymphodepletion or ancillary IL-2.

### Example 5

### Improvement of Cellular Therapies

In this example, Slc16a11 is functionally deleted from a T cell to be used in Adoptive Cell Transfer (ACT) Therapy. A functional deletion can be achieved, for example, by using CRISPR/Cas system or RNAi (e.g., siRNA duplexes or shRNA vectors) to reduce Slc16a11 expression in a T cell. In one example, Cas9 or dCas9 is used to target/edit the Slc16a11 gene in a T cell. In another example, Cas13d, dCas13d, or RNAi is used to target/reduce the Slc16a11 RNA in a T cell.

In one example, CRISPR/Cas (such as Cas9, dCas9, dCas13d, or Cas13) or RNAi is used to functionally delete Slc16a11 (e.g., targeting/editing the gene or targeting/reducing mRNA), in peripheral blood mononuclear cell (PBMC) derived T cell. The resulting Slc16a11 KO T cells are activated and transduced with a vector encoding a chimeric antigen receptor (CAR) that recognizes a tumor-associated antigen. Exemplary tumor targets include, but are not limited to: CD19, BCMA, mesothelin, and MUC1. A subject with a tumor that expresses the tumor-associated antigen is administered a therapeutically effective amount of the Slc16a11 KO CAR-T cells to treat the cancer or tumor.

In another example, CRISPR-Cas9 or RNAi is used to functionally delete Slc16a11 in tumor infiltrating lymphocyte (TIL) T cells that have been isolated from a patient with cancer, for example, isolated from a tumor biopsy of the patient. The Slc16a11 KO TIL T cells are expanded with IL-2, and then a therapeutically effective amount is administered to the patient to treat the cancer or tumor. In yet another example, CRISPR-Cas9 or RNAi is used to functionally delete Slc16a11 in T cell receptor (TCR) engineered T cells (for example, NY-ESO-1). A therapeutically effective amount of the Slc16a11 KO TCR T cells is administered to a patient in need of treating cancer or a tumor.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

1. A modified peripheral blood mononuclear cell (PBMC) with reduced expression of Slc16a11, reduced activity of MCT11, or both, wherein the modified PBMC comprises:
1) an agent that reduces Slc16a11 expression; or
2) a non-naturally occurring genetic modification that reduces an amount of functional MCT11.

2. The modified PBMC of claim 1, comprising the agent that reduces Slc16a11 expression, wherein the agent comprises an inhibitory RNA (RNAi) specific for Slc16a11 or a guide RNA (gRNA) specific for Slc16a11.

3. The modified PBMC of claim 2, wherein the RNAi is a short hairpin RNA (shRNA) molecule, short interfering RNA (siRNA) molecule, or antisense RNA molecule.

4. The modified PBMC of claim 2, wherein the agent comprises:
a) the RNAi specific for Slc16a11, wherein the RNAi specific for Slc16a11 comprises at least 90% sequence identity to a portion of the Slc16a11 gene or transcript, or
b) the gRNA specific for Slc16a11, wherein the gRNA specific for Slc16a11 comprises at least 90% sequence identity to a portion of the Slc16a11 gene or transcript, or
c) the gRNA specific for Slc16a11, wherein the gRNA specific for Slc16a11 comprises at least 90% sequence identity to a portion of the Slc16a11 gene or transcript, and the agent further comprises a Cas nuclease.

5. (canceled)

6. The modified PBMC of claim 4, comprising c) the gRNA specific for Slc16a11 and the Cas nuclease, wherein the Cas nuclease comprises a Cas9, dCas9, Cas13d, or dCas13d nuclease.

7. The modified PBMC of claim 1, wherein the genetic modification is a point mutation, a partial deletion, full deletion, or insertion of Slc16a11 that reduces expression of Slc16a11 and/or reduces activity of MCT11.

8. The modified PBMC of claim 1, wherein the modified PBMC is a T cell.

9-10. (canceled)

11. The modified PBMC of claim 8, wherein the T cell is a tumor-infiltrating lymphocyte (TIL).

12. The modified PBMC of claim 8, wherein the T cell comprises a chimeric antigen receptor (CAR) or engineered T cell receptor (TCR).

13. The modified PBMC of claim 1, wherein the T cell is reactive to a tumor-specific antigen.

14. The modified PBMC of claim 13, wherein the tumor-specific antigen is one or more of CD19, CD20, BCMA, MUC1, PSA, CEA, HER1, HER2, TRP-2, EpCAM, GPC3, mesothelin 1(MSLN), or EGFR.

15. The modified PBMC of claim 8, wherein the T cell is an exhausted T cell.

16. A method of generating the modified PBMC of claim 1, comprising introducing the agent, or non-naturally occurring genetic modification into a PBMC, thereby generating the modified PBMC with reduced expression of Slc16a11, reduced activity of MCT11, or both.

17. The method of claim 16, wherein the PBMC is a T cell.

18. The method of claim 16, wherein the method further comprises incubating the modified PBMC with interleukin 2 (IL-2), interleukin 7 (IL-7), interleukin 15 (IL-15), or combinations thereof.

19-25. (canceled)

26. A pharmaceutical composition comprising:
the modified PBMC of claim 1, and
a pharmaceutically acceptable carrier.

27. (canceled)

28. A method for treating cancer or a tumor in a subject, the method comprising:
administering a therapeutically effective amount of the modified PBMC of claim 1 to the subject having cancer or the tumor, thereby treating the cancer or the tumor.

29. The method of claim 28, wherein the modified PBMC is autologous or allogeneic to the subject.

30. (canceled)

31. The method of claim 28, further comprising administering a IL-2, IL-7, and/or IL-15 to the subject.

32. The method of claim 28, further comprising:
treating the subject with one or more of: surgery, radiation, chemotherapy, biologic therapy, or immunotherapy; or
administering to the subject a therapeutically effective amount of one or more of: a checkpoint inhibitor, a T cell agonist antibody, an oncolytic virus, or an adoptive cell transfer (ACT) immunotherapy.

33. (canceled)

34. The method of claim 28, wherein non-modified lymphocytes are depleted in the subject prior to administering the modified PBMC.

35. The method of claim 28, wherein the cancer or tumor is a leukemia, colorectal cancer, melanoma, cervical cancer, lung cancer, ovarian cancer, bladder cancer, breast cancer, pancreatic cancer, renal cell carcinoma, prostate cancer, or head and neck cancer.


SEQUENCE LISTING (8 sequences)

SEQ ID NO: 1 - protein, 471 aa, Homo sapiens
MPAPQRKHRRGGFSHRCFPTPQTAMTPQPAGPPDGGWGWVVAAAAFAINGLSYGLLRSLGLAFPDLAEHFDRSAQDTAWISALALAVQQAASPVGSALSTRWGARPVVMVGGVLASLGFVFSAFASDLLHLYLGLGLLAGFGWALVFAPALGTLSRYFSRRRVLAVGLALTGNGASSLLLAPALQLLLDTFGWRGALLLLGAITLHLTPCGALLLPLVLPGDPPAPPRSPLAALGLSLFTRRAFSIFALGTALVGGGYFVPYVHLAPHALDRGLGGYGAALVVAVAAMGDAGARLVCGWLADQGWVPLPRLLAVFGALTGLGLWVVGLVPVVGGEESWGGPLLAAAVAYGLSAGSYAPLVFGVLPGLVGVGGVVQATGLVMMLMSLGGLLGPPLSGFLRDETGDFTASFLLSGSLILSGSFIYIGLPRALPSCGPASPPATPPPETGELLPAPQAVLLSPGGPGSTLDTTC

SEQ ID NO: 2 - DNA, 1726 bp, synthetic construct
  source 1..1726: mol_type = other DNA
catttctcgccgctcccccctcccccgggcctgggggttgtgtgtgtacataattcaatccccgtgggactggcgtctggcctcacgcgcggcttgccggattggctgtttcagctcgcccccgccctccgtacaccccgcgtcccaggtggctcaggcccctgtggtgatctctgtttaccgagagagcccgtccaagttgggctccatcgctgccctcgctccccttcggggcccccgcccgcctgggaagcagagagaaagccgggcccagcccttcctcacccttcccctccccgcaccgcccggagaggtcggacggcgatgaccccccagcccgccggacccccggatgggggctggggctgggtggtggcggccgcagccttcgcgataaacgggctgtcctacgggctgctgcgctcgctgggccttgccttccctgaccttgccgagcactttgaccgaagcgcccaggacactgcgtggatcagcgccctggccctggccgtgcagcaggcagccagccccgtgggcagcgccctgagcacgcgctggggggcccgccccgtggtgatggttgggggcgtcctcgcctcgctgggcttcgtcttctcggctttcgccagcgatctgctgcatctctacctcggcctgggcctcctcgctggctttggttgggccctggtgttcgcccccgccctaggcaccctctcgcgttacttctcccgccgtcgagtcttggcggtggggctggcgctcaccggcaacggggcctcctcgctgctcctggcgcccgccttgcagcttcttctcgatactttcggctggcggggcgctctgctcctcctcggcgcgatcaccctccacctcaccccctgtggcgccctgctgctacccctggtccttcctggagaccccccagccccaccgcgtagtcccctagctgccctcggcctgagtctgttcacacgccgggccttctcaatctttgctctaggcacagccctggttgggggcgggtacttcgttccttacgtgcacttggctccccacgctttagaccggggcctggggggatacggagcagcgctggtggtggccgtggctgcgatgggggatgcgggcgcccggctggtctgcgggtggctggcagaccaaggctgggtgcccctcccgcggctgctggccgtattcggggctctgactgggctggggctgtgggtggtggggctggtgcccgtggtgggcggcgaagagagctgggggggtcccctgctggccgcggctgtggcctatgggctgagcgcggggagttacgccccgctggttttcggtgtactccccgggctggtgggcgtcggaggtgtggtgcaggccacagggctggtgatgatgctgatgagcctcggggggctcctgggccctcccctgtcaggcttcctaagggatgagacaggagacttcaccgcctctttcctcctgtctggttctttgatcctctccggcagcttcatctacatagggttgcccagggcgctgccctcctgtggtccagcctcccctccagccacgcctcccccagagacgggggagctgcttcccgctccccaggcagtcttgctgtccccaggaggccctggctccactctggacaccacttgttgattattttcttgtttgagcccctcccccaataaagaatttttatcgggttttcctgaaa

SEQ ID NO: 3 - RNA, 20 nt, synthetic construct
  source 1..20: mol_type = other RNA
cgggggtccggcgggctggg

SEQ ID NO: 4 - RNA, 58 nt, synthetic construct
  source 1..58: mol_type = other RNA
ccgggcagcttcttctcgatactttctcgagaaagtatcgagaagaagctgctttttg

SEQ ID NO: 5 - protein, 447 aa, Mus musculus
MTPKPAGPPDGGWGWVVAAAAFAVNGLSYGLLRSLGLALPDLAEHFERSAQDTAWVSALALAVQQAASPVGSALSTRWGARPVVMVGGVLTSLGLVFSAFARSLLHLYLGLGLLAGSGWALVFAPALGTLSRYFSRRRVLAVGLALTGNGASSLLLAPALQFLLDTFGWRGALLLLGAVTLHLTPCGALLRPLALSGDPLAPPRTPLAALGLGLFKRRAFSVFALGTALIGGGYFVPYVHLGPHALDQGMGGYGAALVVAVAAVGDACARLASGWLADQGWVPLPRLLVVFGSLTGLGVLAMGLVPTVGTEEGWGAPLLAAAGAYGLSAGSYAPLVFGVLPGLVGIGGVVQATGLVMMLMSLGGLLGPPLSGFLRDKTGDFSASFLVCSSFILSGSFIYMGLPRALPSCRPASPPATPPPERGELLPVPQVSLLSAGGTGSIRDTTC

SEQ ID NO: 6 - DNA, 1773 bp, synthetic construct
  source 1..1773: mol_type = other DNA
gaggcggggcgcggccaccaggtacgctcactcctcgcggttccccaaccagggctggggctcgtgtgtggataattcaatcctcgtgggactcggcgtcaggccttagccaggcctgggtggattggctgtctctctttcccccgccctcggtattcacccccacccccaccccgtgtcccaggtggcttgggcccctgtggtgatctctgtttactggagagagcggtccaagttgggctccatctctgtgctggctagctgctccgcagaaccctttctgcggaaagcgcggagaaaatccggccgaacccaccttcgccctttccctacccccaacgccgggagaggtcggcagacggcgatgacccccaagccggccggacccccggacgggggctggggctgggtggtggcggccgcagcattcgccgtgaacgggctctcctacgggctcttacgctccctgggccttgccctccccgacctcgcggagcactttgaacgaagcgcccaggacactgcgtgggtcagcgccctggccttggccgtgcagcaggcagccagcccagtgggcagcgccctgagcactcgctggggggcacgccccgtggtgatggttgggggagtcctaacctcgcttggcttggtcttctcggctttcgcccgaagcctcctgcacctctacctcggcctgggcctcctcgctggctctggctgggccctggtgtttgccccagccctgggtaccctctctcggtacttctcccgccgtcgggtcttggcggtagggttggcgctcaccggtaatggggcatcctcgctgctcctggcacctgccttgcagttcctccttgatactttcggctggcggggtgccttgctcctccttggcgctgtcacccttcacctcacaccctgtggcgccttgctaagacctttagctctctctggtgacccgctggccccacctcgcacccccctagctgcccttggcctaggtctgttcaagcgccgggccttttcagtctttgctttgggcaccgccttgatcgggggcggatacttcgtcccctacgttcatttgggtccgcatgctttagatcaaggcatgggtggttatggggcagcgttagtggtggctgtcgctgcagtgggagatgcctgtgcccgattggccagcggatggctggcagaccagggctgggtgccccttccgaggcttctggtggtgtttgggtctctgactgggttaggggtactagcaatgggactagtgcccactgtggggacagaggagggttggggggctcctctgctggccgctgctggggcctatgggctgagcgctgggagttatgccccactggttttcggtgtgctcccggggctggtgggcattggaggtgtggtgcaggccacagggctggtgatgatgctgatgagcctcgggggactcctgggccctcctctgtcaggcttcctaagggataagacaggagacttcagtgcctctttcctggtgtgcagctctttcatcctctctggcagtttcatctacatggggctgcccagagccctcccctcctgccgtccagcctcacctccagcaacccctccaccagagagaggggagctgctcccagttccacaagtctccctgctttccgcagggggtactggctccatccgggataccacttgttgatcattttcttggttgacctccttccctaataaagaatttttatcttacaaaaaaaaaaaaaaaaa

SEQ ID NO: 7 - RNA, 20 nt, synthetic construct
  source 1..20: mol_type = other RNA
cgcccccttctaggcccagt

SEQ ID NO: 8 - RNA, 21 nt, synthetic construct
  source 1..21: mol_type = other RNA
tggcttggtcttctcggcttt